(19) United States
(12) Patent Application Publication
(10) Pub. No.: US 2026/0078405 A1
(43) Pub. Date: Mar. 19, 2026

(54) GENE THERAPY FOR TREATING CITRULLENEMIA

(71) Applicant: The Trustees of the University of Pennsylvania, Philadelphia, PA (US)

(72) Inventors: James M. Wilson, Philadelphia, PA (US); Jenny Agnes Sidrane, Phoenixville, PA (US); Lili Wang, Phoenixville, PA (US)

(73) Assignee: The Trustees of the University of Pennsylvania, Philadelphia, PA (US)

(21) Appl. No.: 19/296,641

(22) Filed: Aug. 11, 2025

Related U.S. Application Data
(63) Continuation of application No. 18/057,572, filed on Nov. 21, 2022, now Pat. No. 12,385,065.

(51) Int. Cl. C12N 15/86; A61K 48/00; A61P 43/00

(52) U.S. Cl. CPC C12N 15/86; A61P 43/00; C12Y 603/04005; A61K 48/00; C12N 2750/14143; C12N 2830/008; C12N 2830/42

(57) ABSTRACT

Compositions and regimens useful in treating type I citrullenemia are provided. The compositions include recombinant adeno-associated virus (rAAV) with a transthyretin enhancer and promoter driving expression of a human Argininosuccinate Synthase 1 (ASS1).

### CROSS-REFERENCE TO RELATED APPLICATIONS

This is a continuation of U.S. patent application Ser. No. 18/057,572, filed Nov. 21, 2022, which is a continuation of U.S. patent application Ser. No. 16/478,971, filed Jul. 18, 2019, now U.S. Pat. No. 11,535,866, granted Dec. 27, 2022, which is a national stage application under 35 USC 371 of PCT/US2018/061413, filed Jan. 2, 2018, which claims the benefit under 35 USC 119 (e) of U.S. Provisional Patent Application No. 62/453,424, filed Feb. 1, 2017, and US Provisional Patent Application No. 62/469,650, filed Mar. 10, 2017. Each of these applications is hereby incorporated by reference in its entirety.

### INCORPORATION-BY-REFERENCE OF MATERIAL SUBMITTED IN ELECTRONIC FORM

Applicant hereby incorporates by reference the Sequence Listing material filed in electronic form herewith. This file is labeled “16-7938USC1.xml” (created Feb. 19, 2025 and 139,685 bytes in size).

### 1. BACKGROUND

The application relates to embodiments useful for a gene therapy for treating type I citrullenemia. Type I citrullenemia is an autosomal recessive disease caused by mutations in argininosuccinate synthase 1 (ASS1) enzyme that catalyzes the synthesis of argininosuccinate from citrulline and aspartate, resulting in citrullinemia and buildup of ammonia.

The clinical spectrum of Type I citrullenemia (CTLN1) ranges from severe neonatal onset form to milder late-onset forms. Owing to its relatively recent addition to the newborn screening panel, patients with type I citrullinemia will be identified early, allowing immediate implementation of treatment. However, despite this early identification of disease and treatment, some patients may progress. The untreated mortality rate in untreated classical CTLN1 is 100%, with most deaths occurring before 17 days of life.

Current treatment approaches for type I citrullenemia include dietary restriction (restriction of protein intake), medications (nitrogen scavenger therapy and carnitine), and arginine supplementation. Liver transplantation is curative for citrullinemia but transplant recipients are required to maintain a constant immune suppression regimen to prevent rejection. Liver directed AAV treatments for type I citrullenemia have been shown. See, e.g., Chandler et al, Liver-directed adeno-associated virus serotype 8 gene transfer rescues a lethal murine model of citrullinemia type 1, Gene Therapy (2013) 20, 1188-1191, which is incorporated herein by reference. However, liver-directed gene therapy did not fully correct the biochemical phenotype of systemic ASS1 deficiency; arginine levels plummeted in treated individuals of a murine model for CTLN1 (fold/fold) due to persistent renal deficiency. See also, Kok et al, Adeno-associated Virus-mediated Rescue of Neonatal Lethality in Argininosuccinate Synthase-deficient Mice, Molecular Therapy vol. 21 no. 10, 1823-1831 October 2013, which is incorporated herein by reference.

What are needed are more effective treatments for type 1 citrullinemia.

### 2. SUMMARY

The embodiments described herein relate to an AAV gene therapy vector for delivering normal human Argininosuccinate Synthase 1 (ASS1) to a subject in need thereof, following intravenous administration of the vector resulting in long-term, perhaps 10 years or more, of clinically meaningful correction of Type I citrullenemia (CTLN1) (also sometimes called citrullinuria or ASS1 deficiency). The subject patient population is patients with moderate to severe Type I citrullenemia, including those with the acute neonatal form (the “classic” form), a milder late-onset form (the “non-classic” form), or the form in which women have onset of severe symptoms during pregnancy or post-partum. The intended vector dose is, in one embodiment, intended to deliver ASS1 which results in near normal citrulline, glutamine and ammonia plasma levels. However, even nominal reductions in citrulline, glutamine and ammonia levels are desirable, and a desirable endpoint. As reported by Quinonez and Thoene, Pagon R A, Adam M P, Ardinger H H, et al., editors. Seattle (WA): University of Washington, Seattle; 1993-2016 (incorporated herein by reference), elevation of either citrulline or ammonia above acceptable levels (ammonia >100 μmol/L or plasma citrulline >˜100 μmol/L) is sufficient evidence to initiate treatment for CTLN1. In another embodiment, a neonatal diagnosis based on genetic testing is sufficient to initiate treatment.

In one aspect, this application provides the use of a replication deficient adeno-associated virus (AAV) to deliver a human Argininosuccinate Synthase 1 (hASS1) gene to liver cells of patients (human subjects) diagnosed with CTLN1. The recombinant AAV vector (rAAV) used for delivering the hASS1 gene (“rAAV.hASS1”) should have a tropism for the liver (e.g., a rAAV bearing an AAV8 capsid), and the hASS1 transgene should be controlled by liver-specific expression control elements. In one embodiment, the expression control elements include one or more of the following: an enhancer; a promoter; an intron; a WPRE; and a polyA signal. Such elements are further described herein.

In one embodiment, the ASS1 protein sequence is shown in SEQ ID NO: 1. In one embodiment, the hASS1 coding sequence is shown in SEQ ID NO: 3. The coding sequence for hASS1 is, in one embodiment, codon optimized for expression in humans. Such sequence may share at least 80% identity to the native hASS1 coding sequence (SEQ ID NO: 3). In another embodiment, the hASS1 coding sequence is that shown in SEQ ID NO: 3.

In another aspect, provided herein is an aqueous suspension suitable for administration to a CTLN1 patient which includes the rAAV described herein. In some embodiments, the suspension includes an aqueous suspending liquid and about 1×10¹² to about 1×10¹⁴ genome copies (GC) of the rAAV/mL. The suspension is, in one embodiment, suitable for intravenous injection. In other embodiment, the suspension further includes a surfactant, preservative, and/or buffer dissolved in the aqueous suspending liquid.

In another embodiment, provided herein is a method of treating a patient having CTLN1 with an rAAV as described herein. In one embodiment, about 1×10¹¹ to about 3×10¹³ genome copies (GC) of the rAAV/kg patient body weight are delivered the patient in an aqueous suspension.

### 3. BRIEF DESCRIPTION OF THE DRAWINGS

FIG. 1 is a schematic representation of AAV.hASS1co cis plasmid.

FIG. 2A is a survival curve of ASS1^{fold/fold} mice as described in Example 2. Squares are male mice and circles are female mice.

FIG. 2B is a line graph of body weights in both genders (M, male; F, female) of ASS1^{fold/fold} mice (Fold). Wild-type littermates (WT) were provided as controls.

FIG. 2C is a graph showing elevated plasma NH₃ levels of ASS1^{fold/fold} mice. Wild-type (WT) mice were provided as controls. Each circle or triangle indicates one sample. Mean±SEM is also plotted. Mann Whitney test was performed and the p value between indicated groups is shown in the figure.

FIG. 2D is a graph showing elevated plasma citrulline levels of ASS1^{fold/fold} mice. Wild-type (WT) mice were provided as controls. Each circle or triangle indicates one sample. Mean±SEM is also plotted. Mann Whitney test was performed and the p value between indicated groups is shown in the figure.

FIG. 2E is a graph showing elevated plasma arginine levels of ASS1^{fold/fold} mice. Wild-type (WT) mice were provided as controls. Each circle or triangle indicates one sample. Mean±SEM is also plotted. Mann Whitney test was performed and the p value between indicated groups is shown in the figure.

FIG. 2F is a graph showing elevated urine orotic acid levels of ASS1^{fold/fold} mice at all age, or at the age of 4 weeks, 5 weeks, 6 weeks, 8 weeks, 10 weeks, 12 weeks and 14 weeks and older. Wild-type (WT) mice were provided as controls. Each dot indicates one sample. Mean±SEM is also plotted.

FIG. 3A is a line graph of body weights of male ASS1^{fold/fold} mice injected intravenously at birth with 3×10¹¹ GC/pup of AAV8.TBG.PI.hASS1co. WPRE.bGH (Fold-M-G2, solid gray square, n=1) or AAV8.LSP.IVS2.hASS1co.bGH (Fold-M-G3, asterisk, n=3) or AAV8.TBG.PI.hASS1co.bGH (Fold-M-G4, triangle, n=3). Male ASS1^{fold/fold} (Fold-M, open square) and wild-type (WT-M, solid black square) mice without treatment were provided as controls.

FIG. 3B is a line graph of body weights of female ASS1^{fold/fold} mice injected intravenously at birth with 3×10¹¹ GC/pup of AAV8.TBG.PI.hASS1co. WPRE.bGH (Fold-F-G2, solid grey circle, n=1) or AAV8.LSP.IVS2.hASS1co.bGH (Fold-F-G3, asterisk, n=2) or AAV8.TBG.PI.hASS1co.bGH (Fold-F-G4, triangle, n=1). Female ASS1^{fold/fold} (Fold-F, open circle) and wild-type (WT-F, solid black circle) mice without treatment were provided as controls.

FIG. 3C is a line graph of body weights of male ASS1^{fold/fold} mice injected intravenously at birth with 1×10¹¹ GC/pup of AAV8.TBG.PI.hASS1co. WPRE.bGH (Fold-M-G2, red, n=2) or AAV8.LSP.IVS2.hASS1co.bGH (Fold-M-G3, purple, n=1). Male ASS1^{fold/fold} (Fold-M, blue) and wild-type (WT-M, black) mice without treatment were provided as controls.

FIG. 3D is a line graph of body weights of female ASS1^{fold/fold} mice injected intravenously at birth with 1×10¹¹ GC/pup of AAV8.TBG.PI.hASS1co. WPRE.bGH (Fold-F-G2, red, n=1) or AAV8.LSP.IVS2.hASS1co.bGH (Fold-F-G3, purple, n=2) or AAV8.TBG.PI.hASS1co.bGH (Fold-F-G4, green, n=2). Female ASS1^{fold/fold} (Fold-F, blue) and wild-type (WT-F, black) mice without treatment were provided as controls.

FIG. 3E is a line graph of body weights of male ASS1^{fold/fold} mice injected intraperitoneal on postnatal day 14 with 1×10¹¹ GC/mouse of AAV8.TBG.PI.hASS1co.WPRE.bGH (Fold-M-IP d14, light blue, n=4). Male ASS1^{fold/fold} (Fold-M, blue) and wild-type (WT-M, black) mice without treatment were provided as controls.

FIG. 3F is a line graph of body weights of female ASS1^{fold/fold} mice injected intraperitoneal on postnatal day 14 with 1×10¹¹ GC/mouse of AAV8.TBG.PI.hASS1co. WPRE.bGH (Fold-F-IP d14, magenta, n=2). Female ASS1^{fold/fold} (Fold-F, blue) and wild-type (WT-F, black) mice without treatment were provided as controls.

FIG. 4A is a bar graph of citrulline levels in the blood of ASS1^{fold/fold} mice injected intravenously at birth with 3×10¹¹ GC/pup of AAV8.TBG.PI.hASS1co.WPRE.bGH (G2-6w) or AAV8.LSP.IVS2.hASS1co.bGH (G3-6w) or AAV8.TBG.PI.hASS1co.bGH (G4-6w). ASS1^{fold/fold} (Fold) and wild-type (WT-M) mice without treatment were provided as controls.

FIG. 4B is a bar graph of citrulline levels in the blood of ASS1^{fold/fold} mice injected intravenously at birth with 1×10¹¹ GC/pup of AAV8.TBG.PI.hASS1co.WPRE.bGH (G2B-12w) or AAV8.LSP.IVS2.hASS1co.bGH (G3B-6w) or AAV8.TBG.PI.hASS1co.bGH (G4B-6w). ASS1^{fold/fold} (Fold) and wild-type (WT-M) mice without treatment were provided as controls.

FIG. 5A is a line graph of urine orotic acid levels in ASS1^{fold/fold} mice injected intravenously at birth with 3×10¹¹ GC/pup of AAV8.TBG.PI.hASS1co. WPRE.bGH. Data from mice with identification number 028 and 031 was acquired and plotted.

FIG. 5B is a line graph of urine orotic acid levels in ASS1^{fold/fold} mice injected intravenously at birth with 3×10¹¹ GC/pup of AAV8.LSP.IVS2.hASS1co.bGH. Data from mice with identification number 022, 038, 018 and 001 was acquired and plotted.

FIG. 5C is a line graph of urine orotic acid levels in ASS1^{fold/fold} mice injected intravenously at birth with 3×10¹¹ GC/pup of AAV8.TBG.PI.hASS1co.bGH. Data from mice with identification number 024, 025 and 043 was acquired and plotted.

FIG. 5D is a line graph of urine orotic acid levels in ASS1^{fold/fold} mice injected intravenously at birth with 1×10¹¹ GC/pup of AAV8.LSP.IVS2.hASS1co.bGH. Data from mice with identification number 059, 069 and 070 was acquired and plotted.

FIG. 6A is a survival curve of ASS1^{fold/fold} mice demonstrating liver-directed gene therapy improves the survival of ASS1^{fold/fold} Four-week old ASS1^{fold/fold} (both males and females) received a single retro-orbital injection of AAV8.LSP.IVS.hASS1co.bGH vector at the dose of 3×10¹¹ GC/mouse (n=10) or 1×10¹¹ GC/mouse (n=10). Survival was monitored. Untreated ASS1^{fold/fold} mice were provided as controls (n=24).

FIG. 6B is a line graph of body weights of male ASS1^{fold/fold} mice demonstrating liver-directed gene therapy improves the body weight of ASS1^{fold/fold} Four-week old ASS1^{fold/fold} received a single retro-orbital injection of AAV8.LSP.IVS.hASS1co.bGH vector at the dose of 3×10¹¹ GC/mouse or 1×10¹¹ GC/mouse. Body weights were monitored. Gender-matched untreated ASS1^{fold/fold} (Fold) and wild-type (WT) mice were provided as controls.

FIG. 6C is a line graph of body weights of female ASS1^{fold/fold} mice demonstrating liver-directed gene therapy improves the body weight of ASS1^{fold/fold} Four-week old ASS1^{fold/fold} received a single retro-orbital injection of AAV8.LSP.IVS.hASS1co.bGH vector at the dose of 3×10¹¹ GC/mouse or 1×10¹¹ GC/mouse. Body weights were monitored. Gender-matched untreated ASS1^{fold/fold} (Fold) and wild-type (WT) mice were provided as controls.

FIG. 7A is a line graph of body weights of male ASS1^{fold/fold} mice demonstrating liver-directed gene therapy improves the body weight of ASS1^{fold/fold} Four-week old ASS1^{fold/fold} received a single retro-orbital injection of AAV8.ApoE.A1AT (full).IVS2.hASS1co.bGH vector at the dose of 3×10¹¹ GC/mouse or 1×10¹¹ GC/mouse. Body weights were monitored. Gender-matched untreated ASS1^{fold/fold} (Fold) and wild-type (WT) mice were provided as controls.

FIG. 7B is a line graph of body weights of female ASS1^{fold/fold} mice demonstrating liver-directed gene therapy improves the body weight of ASS1^{fold/fold} Four-week old ASS1^{fold/fold} received a single retro-orbital injection of AAV8.ApoE.A1AT (full).IVS2.hASS1co.bGH vector at the dose of 3×10¹¹ GC/mouse or 1×10¹¹ GC/mouse. Body weights were monitored. Gender-matched untreated ASS1^{fold/fold} (Fold) and wild-type (WT) mice were provided as controls.

FIG. 8 is a line graph showing reduction of urine orotic acid in ASS1^{fold/fold} following vector administration. Four-week old ASS1^{fold/fold} received a single retro-orbital injection of AAV8.LSP.IVS2.hASS1co.bGH vector at the dose of 3×10¹¹ or 1×10¹¹ GC/mouse. ASS1^{fold/fold} (Fold) mice were provided as controls.

FIGS. 9A to 9B provide graphs showing citrulline levels in ASS1^{fold/fold} mice at 2 weeks and 6 weeks post AAV8 vector administration. Four-week old ASS1^{fold/fold} received a single retro-orbital injection of the indicated vector at the dose of 3×10¹¹ GC/mouse (FIG. 9A) or 1×10¹¹ GC/mouse (FIG. 9B). Code for each vector is listed in Table 1. Untreated ASS1^{fold/fold} (Fold) and wild-type (WT) mice were provided as controls. Significant differences compared to untreated fold mice were calculated using one-way ANOVA Dunnett's multiple comparisons test. *P<0.05, **P<0.01, ***P<0.001, ****P<0.0001.

FIG. 10 is a graph showing percent survival in ASS1^{fold/fold} mice. Four-week old ASS1^{fold/fold} received a single retro-orbital injection of the indicated vector at the dose of 1×10¹¹ GC/mouse. Code for each vector is listed in Table 1. Untreated ASS1^{fold/fold} (Fold) and wild-type (WT) mice were provided as controls.

FIG. 11A is a graph showing body weight in male ASS1^{fold/fold} mice. Four-week old ASS1^{fold/fold} received a single retro-orbital injection of the indicated vector at the dose of 1×10¹¹ GC/mouse. Code for each vector is listed in Table 1. Untreated ASS1^{fold/fold} (Fold) and wild-type (WT) mice were provided as controls. Liver-directed gene therapy improves the body weight of ASS1^{fold/fold}.

FIG. 11B is a graph showing body weight in female ASS1^{fold/fold} mice. Four-week old ASS1^{fold/fold} received a single retro-orbital injection of the indicated vector at the dose of 1×10¹¹ GC/mouse. Code for each vector is listed in Table 1. Untreated ASS1^{fold/fold} (Fold) and wild-type (WT) mice were provided as controls. Liver-directed gene therapy improves the body weight of ASS1^{fold/fold}.

FIGS. 12A and 12B are graphs showing a reduction in plasma citrulline levels by AAV. Four-week old ASS1 fold/fold (both males and females) received a single retro-orbital injection of AAV8-hASS1 vectors at the dose of 1×10¹¹ GC/mouse. Code for each vector is listed in Table 1. Plasma citrulline levels at 2 weeks (A) and 6 weeks (B) post vector administration. **P<0.01; ****P<0.0001, one-way ANOVA, Kruskal-Wallis test, compared to untreated fold mice.

FIG. 13 is a graph showing a reduction in plasma citrulline levels by AAV. Four-week-old fold mice received a single retro orbital injection of AAV8-hASS1 vectors at the dose of 1.0×10¹¹ GC. Plasma NH3 levels at 6 weeks post vector injection are shown here. **P<0.01; ***P<0.001; ****P<0.0001, one-way ANOVA, Kruskal-Wallis test, compared to untreated fold mice.

### 4. DETAILED DESCRIPTION

The embodiments described in the application relate to the use of a replication deficient adeno-associated virus (AAV) to deliver a human Argininosuccinate Synthase 1 (hASS1) gene to liver cells of patients (human subjects) diagnosed with type 1 citrullenemia (CTLN1). The recombinant AAV vector (rAAV) used for delivering the hASS gene (“rAAV.hASS1”) should have a tropism for the liver (e.g., an rAAV bearing an AAV8 capsid), and the hASS1 transgene should be controlled by liver-specific expression control elements. In one embodiment, the expression control elements include one or more of the following: an enhancer; a promoter; an intron; a WPRE; and a polyA signal. Such elements are further described herein.

As used herein, “AAV8 capsid” refers to the AAV8 capsid having the amino acid sequence of GenBank, accession: YP_077180.1, SEQ ID NO: 19, and/or an AAV8 capsid encoded by the nucleic acid sequence of GenBank: AF513852.1, nt 2121-4337, SEQ ID NO: 36, which sequences are incorporated by reference herein. Some variation from this encoded sequence is permitted, which may include sequences having about 99% identity to the referenced amino acid sequence in YP_077180.1 and WO 2003/052051 (which is incorporated herein by reference) (i.e., less than about 1% variation from the referenced sequence). Methods of generating the capsid, coding sequences therefore, and methods for production of rAAV viral vectors have been described. See, e.g., Gao, et al, Proc. Natl. Acad. Sci. U.S.A. 100 (10), 6081-6086 (2003) and US 2015/0315612.

As used herein, the term “NAb titer” a measurement of how much neutralizing antibody (e.g., anti-AAV Nab) is produced which neutralizes the physiologic effect of its targeted epitope (e.g., an AAV). Anti-AAV NAb titers may be measured as described in, e.g., Calcedo, R., et al., Worldwide Epidemiology of Neutralizing Antibodies to Adeno-Associated Viruses. Journal of Infectious Diseases, 2009. 199 (3): p. 381-390, which is incorporated by reference herein.

The terms “percent (%) identity”, “sequence identity”, “percent sequence identity”, or “percent identical” in the context of amino acid sequences refers to the residues in the two sequences which are the same when aligned for correspondence. Percent identity may be readily determined for amino acid sequences over the full-length of a protein, polypeptide, about 32 amino acids, about 330 amino acids, or a peptide fragment thereof or the corresponding nucleic acid sequence coding sequencers. A suitable amino acid fragment may be at least about 8 amino acids in length, and may be up to about 700 amino acids. Generally, when referring to “identity”, “homology”, or “similarity” between two different sequences, “identity”, “homology” or “similarity” is determined in reference to “aligned” sequences. “Aligned” sequences or “alignments” refer to multiple nucleic acid sequences or protein (amino acids) sequences, often containing corrections for missing or additional bases or amino acids as compared to a reference sequence. Alignments are performed using any of a variety of publicly or commercially available Multiple Sequence Alignment Programs. Sequence alignment programs are available for amino acid sequences, e.g., the “Clustal X”, “MAP”, “PIMA”, “MSA”, “BLOCKMAKER”, “MEME”, and “Match-Box” programs. Generally, any of these programs are used at default settings, although one of skill in the art can alter these settings as needed. Alternatively, one of skill in the art can utilize another algorithm or computer program which provides at least the level of identity or alignment as that provided by the referenced algorithms and programs. See, e.g., J. D. Thomson et al, Nucl. Acids. Res., “A comprehensive comparison of multiple sequence alignments”, 27 (13): 2682-2690 (1999).

As used herein, the term “operably linked” refers to both expression control sequences that are contiguous with the gene of interest and expression control sequences that act in trans or at a distance to control the gene of interest.

A “replication-defective virus” or “viral vector” refers to a synthetic or artificial viral particle in which an expression cassette containing a gene of interest is packaged in a viral capsid or envelope, where any viral genomic sequences also packaged within the viral capsid or envelope are replication-deficient; i.e., they cannot generate progeny virions but retain the ability to infect target cells. In one embodiment, the genome of the viral vector does not include genes encoding the enzymes required to replicate (the genome can be engineered to be “gutless”-containing only the transgene of interest flanked by the signals required for amplification and packaging of the artificial genome), but these genes may be supplied during production. Therefore, it is deemed safe for use in gene therapy since replication and infection by progeny virions cannot occur except in the presence of the viral enzyme required for replication.

It is to be noted that the term “a” or “an” refers to one or more. As such, the terms “a” (or “an”), “one or more,” and “at least one” are used interchangeably herein.

The words “comprise”, “comprises”, and “comprising” are to be interpreted inclusively rather than exclusively. The words “consist”, “consisting”, and its variants, are to be interpreted exclusively, rather than inclusively. While various embodiments in the specification are presented using “comprising” language, under other circumstances, a related embodiment is also intended to be interpreted and described using “consisting of” or “consisting essentially of” language.

As used herein, the term “about” means a variability of 10% from the reference given, unless otherwise specified.

Unless defined otherwise in this specification, technical and scientific terms used herein have the same meaning as commonly understood by one of ordinary skill in the art and by reference to published texts, which provide one skilled in the art with a general guide to many of the terms used in the present application.

### 4.1 Gene Therapy Vectors

In one aspect, a recombinant adeno-associated virus (rAAV) vector carrying the human ASS1 gene is provided for use in gene therapy. The rAAV.hASS1 vector should have a tropism for the liver (e.g., a rAAV bearing an AAV8 capsid) and the hASS1 transgene should be controlled by liver-specific expression control elements. In another embodiment, the rAAV.hASS1 vector has a tropism for kidney. The vector is formulated in a buffer/carrier suitable for infusion in human subjects. The buffer/carrier should include a component that prevents the rAAV from sticking to the infusion tubing but does not interfere with the rAAV binding activity in vivo.

4.1.1. The rAAV.hASS Vector
4.1.1.1. The hASS1 Sequence

Citrullinemia type I (CTLN1) (also called “classic citrullinemia”) results from deficiency of the enzyme argininosuccinate synthase 1 (ASS1), the third step in the urea cycle, in which citrulline is condensed with aspartate to form arginosuccinic acid.

Type I citrullinemia shows kinetically abnormal ASS1 in the liver, kidney, and cultured fibroblasts. In quantitative-type citrullinemia, low ASS1 is found in the liver but not in kidney or cultured skin fibroblasts. Residual enzyme in the liver has normal kinetic properties (Saheki et al., 1981). In a study of mRNA coding for ASS1, Kobayashi et al. (Am J. Hum. Genet., 38:667-80, 1986, which is incorporated herein by reference) found that patients with the quantitative type of citrullinemia had, as demonstrated in previous studies, about 10% of the control value of the enzyme in the liver but a normal level of mRNA. They concluded that in quantitative-type citrullinemia, the decrease in the enzyme protein is due either to increased degradation of the enzyme or to decreased or inhibited translation in the liver.

Although certain pathogenic variants are identified with some phenotypes, the phenotype cannot be predicted in all instances. Severe, classic citrullinemia type I typically results from 22 defined pathogenic variants (Engel et al, Human Mutation, 2009 March; 30 (3): 300-7, which is incorporated herein by reference). The pathogenic variant in exon 15, p.Gly390Arg, remains the most prevalent associated with the classic phenotype. Mild (i.e., late-onset) citrullinemia type I is associated with 12 pathogenic variants.

One goal of therapies described herein would provide functional ASS1 enzyme resulting in citrulline, glutamine, and/or ammonia levels less than 100 μmol/L. In another embodiment, any reduction in citrulline, glutamine, and/or ammonia levels is desirable. Other suitable clinical outcomes may include reduction in the use of scavenger, less restrictive diet or no need for liver transplant.

In one embodiment, the “subject” or “patient” is a mammalian subject having CTLN1 as described above. It is intended that a patient having CTLN1 of any severity is the intended subject. In addition, it is intended that a patient having any mutation in their native ASS1 gene is the intended subject.

In one embodiment, the hASS1 gene encodes the hASS1 protein shown in SEQ ID NO: 1. Thus, in one embodiment, the hASS1 transgene can include, but is not limited to, the sequence provided by SEQ ID NO:2 or SEQ ID NO: 3 which are provided in the attached Sequence Listing, which is incorporated by reference herein. SEQ ID NO: 3 provides the cDNA for native human ASS1. SEQ ID NO: 2 provides an engineered cDNA for human ASS1, which has been codon optimized for expression in humans (sometimes referred to herein as hASS1co). It is to be understood that reference to hASS1 herein may, in some embodiments, refer to the hASS1 native or codon optimized sequence. Alternatively or additionally, web-based or commercially available computer programs, as well as service based companies may be used to back translate the amino acid sequences to nucleic acid coding sequences, including both RNA and/or cDNA. See, e.g., backtranseq by EMBOSS,/www.ebi.ac.uk/Tools/st/; Gene Infinity (www.geneinfinity.org/sms-/sms_backtranslation.html); ExPasy (www.expasy.org/tools/). It is intended that all nucleic acids encoding the described hASS1 polypeptide sequences are encompassed, including nucleic acid sequences which have been optimized for expression in the desired target subject (e.g., by codon optimization).

In one embodiment, the nucleic acid sequence encoding hASS1 shares at least 95% identity with the native hASS1 coding sequence of SEQ ID NO: 3. In another embodiment, the nucleic acid sequence encoding hASS1 shares at least 90, 85, 80, 75, 70, or 65% identity with the native hASS1 coding sequence of SEQ ID NO: 3. In one embodiment, the nucleic acid sequence encoding hASS1 shares about 84% identity with the native hASS1 coding sequence of SEQ ID NO: 3. In one embodiment, the nucleic acid sequence encoding hASS1 is SEQ ID NO: 2.

In one embodiment, the hASS1 coding sequence is codon optimized for expression in the desirable subject species, e.g., humans. Codon-optimized coding regions can be designed by various different methods. This optimization may be performed using methods which are available on-line (e.g., GeneArt,), published methods, or a company which provides codon optimizing services, e.g., as DNA2.0 (Menlo Park, CA). One codon optimizing approach is described, e.g., in International Patent Publication No. WO 2015/012924, which is incorporated by reference herein. See also, e.g., US Patent Publication No. 2014/0032186 and US Patent Publication No. 2006/0136184. Suitably, the entire length of the open reading frame (ORF) for the product is modified. However, in some embodiments, only a fragment of the ORF may be altered (e.g., one or more of the individual immunoglobulin domains). By using one of these methods, one can apply the frequencies to any given polypeptide sequence, and produce a nucleic acid fragment of a codon-optimized coding region which encodes the polypeptide.

A number of options are available for performing the actual changes to the codons or for synthesizing the codon-optimized coding regions designed as described herein. Such modifications or synthesis can be performed using standard and routine molecular biological manipulations well known to those of ordinary skill in the art. In one approach, a series of complementary oligonucleotide pairs of 80-90 nucleotides each in length and spanning the length of the desired sequence are synthesized by standard methods. These oligonucleotide pairs are synthesized such that upon annealing, they form double stranded fragments of 80-90 base pairs, containing cohesive ends, e.g., each oligonucleotide in the pair is synthesized to extend 3, 4, 5, 6, 7, 8, 9, 10, or more bases beyond the region that is complementary to the other oligonucleotide in the pair. The single-stranded ends of each pair of oligonucleotides are designed to anneal with the single-stranded end of another pair of oligonucleotides. The oligonucleotide pairs are allowed to anneal, and approximately five to six of these double-stranded fragments are then allowed to anneal together via the cohesive single stranded ends, and then they ligated together and cloned into a standard bacterial cloning vector, for example, a TOPO® vector available from Thermo Fisher Scientific Inc. The construct is then sequenced by standard methods. Several of these constructs consisting of 5 to 6 fragments of 80 to 90 base pair fragments ligated together, i.e., fragments of about 500 base pairs, are prepared, such that the entire desired sequence is represented in a series of plasmid constructs. The inserts of these plasmids are then cut with appropriate restriction enzymes and ligated together to form the final construct. The final construct is then cloned into a standard bacterial cloning vector, and sequenced. Additional methods would be immediately apparent to the skilled artisan. In addition, gene synthesis is readily available commercially.

4.1.1.2. The rAAV Vector

Because ASS1 is natively expressed in the liver, it is desirable to use an AAV which shows tropism for liver. In one embodiment, the AAV supplying the capsid is AAV8. In another embodiment, the AAV supplying the capsid is AAVrh.10. In yet another embodiment, the AAV supplying the capsid is a Clade E AAV. Such AAV include rh.2; rh.10; rh.25; bb.1, bb.2, pi.1, pi.2, pi.3, rh.38, rh.40, rh.43, rh.49, rh.50, rh.51, rh.52, rh.53, rh.57, rh.58, rh.61, rh.64, hu.6, hu. 17, hu.37, hu.39, hu.40, hu.41, hu.42, hu.66, and hu.67. This clade further includes modified rh.2; modified rh.58; and modified rh.64. See, WO 2005/033321, which is incorporated herein by reference. However, any of a number of rAAV vectors with liver tropism can be used. In another embodiment, the rAAV vector has a tropism for kidney.

In a specific embodiment described in the Examples, infra, the gene therapy vector is an AAV8 vector expressing an hASS1 transgene under control of a thyroxine binding globulin (TBG) promoter referred to as AAV8.TBG.PI.hASS1co. WPRE.bGH. In another embodiment, the WPRE is removed. In another embodiment, the gene therapy vector is an AAV8 vector expressing an hASS1 transgene under control of a A1AT promoter, with an ApoE1 enhancer referred to as AAV8.ApoE.A1AT (full).IVS2.hASS1co.bGH. The external AAV vector component is a serotype 8, T=1 icosahedral capsid consisting of 60 copies of three AAV viral proteins, VP1, VP2, and VP3, at a ratio of 1:1:10. The capsid contains a single-stranded DNA rAAV vector genome.

In one embodiment, the rAAV.hASS1 genome contains an hASS1 transgene flanked by two AAV inverted terminal repeats (ITRs). In one embodiment, the hASS1 transgene includes one or more of an enhancer, promoter, an intron, a Woodchuck Hepatitis Virus (WHP) Posttranscriptional Regulatory Element (WPRE), an hASS1 coding sequence, and polyadenylation (polyA) signal. These control sequences are “operably linked” to the hASS1 gene sequences. The expression cassette containing these sequences may be engineered onto a plasmid which is used for production of a viral vector.

The ITRs are the genetic elements responsible for the replication and packaging of the genome during vector production and are the only viral cis elements required to generate rAAV. The minimal sequences required to package the expression cassette into an AAV viral particle are the AAV 5′ and 3′ ITRs, which may be of the same AAV origin as the capsid, or which of a different AAV origin (to produce an AAV pseudotype). In one embodiment, the ITR sequences from AAV2, or the deleted version thereof (ΔITR), are used. However, ITRs from other AAV sources may be selected. Where the source of the ITRs is from AAV2 and the AAV capsid is from another AAV source, the resulting vector may be termed pseudotyped. Typically, an expression cassette for an AAV vector comprises an AAV 5′ ITR, the hASS1 coding sequences and any regulatory sequences, and an AAV 3′ ITR. However, other configurations of these elements may be suitable. A shortened version of the 5′ ITR, termed ΔITR, has been described in which the D-sequence and terminal resolution site (trs) are deleted. In other embodiments, the full-length AAV 5′ and 3′ ITRs are used. In one embodiment, the 5′ ITR is that shown in SEQ ID NO: 16. In one embodiment, the 3′ ITR is that shown in SEQ ID NO: 17.

Exemplary production plasmids to generate rAAVs are shown in SEQ ID NOs: 22 to 35. In one embodiment, provided herein is the plasmid of SEQ ID NO: 22. In another embodiment, provided herein is the plasmid of SEQ ID NO: 23. In another embodiment, provided herein is the plasmid of SEQ ID NO: 24. In another embodiment, provided herein is the plasmid of SEQ ID NO: 25. In another embodiment, provided herein is the plasmid of SEQ ID NO: 26. In another embodiment, provided herein is the plasmid of SEQ ID NO: 27. In another embodiment, provided herein is the plasmid of SEQ ID NO: 28. In another embodiment, provided herein is the plasmid of SEQ ID NO: 29. In another embodiment, provided herein is the plasmid of SEQ ID NO: 30. In another embodiment, provided herein is the plasmid of SEQ ID NO: 31. In another embodiment, provided herein is the plasmid of SEQ ID NO: 32. In another embodiment, provided herein is the plasmid of SEQ ID NO: 33. In another embodiment, provided herein is the plasmid of SEQ ID NO: 34. In another embodiment, provided herein is the plasmid of SEQ ID NO: 35.

Expression of the hASS1 coding sequence is driven from a liver-specific promoter. An illustrative plasmid and vector described herein uses the thyroxine binding globulin (TBG) promoter (SEQ ID NO: 9), or a modified version thereof. One modified version of the TBG promoter is a shortened version, termed TBG-S1. A modified thyroxine binding globulin (TBG-S1) promoter sequence is shown in SEQ ID NO: 8. Alternatively, other liver-specific promoters may be used such as the transthyretin promoter (TTR) (SEQ ID NO: 11). Another suitable promoter is the alpha 1 anti-trypsin (A1AT), or a modified version thereof (which sequence is shown in SEQ ID NO: 10). In one embodiment, the promoter is an A1AT promoter combined with an ApoE enhancer, sometimes referred to as ApoE.A1AT (full). In one embodiment, the sequence is shown in SEQ ID NO: 20. Another suitable promoter is the Liver specific promoter LSP (TH-binding globulin promoter/alpha1-microglobulin/bikunin enhancer) (SEQ ID NO: 21). Other suitable promoters include human albumin (Miyatake et al., J. Virol., 71:5124 32 (1997)), humAlb; and hepatitis B virus core promoter, (Sandig et al., Gene Ther., 3:1002-9 (1996). See, e.g., The Liver Specific Gene Promoter Database, Cold Spring Harbor, rulai.schl.edu/LSPD, which is incorporated by reference. Although less desired, other promoters, such as viral promoters, constitutive promoters, regulatable promoters [see, e.g., WO 2011/126808 and WO 2013/04943], or a promoter responsive to physiologic cues may be used may be utilized in the vectors described herein.

In one embodiment, the expression control sequences include one or more enhancer. In one embodiment, the En34 enhancer is included (34 bp core enhancer from the human apolipoprotein hepatic control region), which is shown in SEQ ID NO: 4. In another embodiment, the EnTTR (100 bp enhancer sequence from transthyretin) is included. Such sequence is shown in SEQ ID NO: 5. See, Wu et al, Molecular Therapy, 16 (2): 280-289, February 2008, which is incorporated herein by reference. In yet another embodiment, the α1-microglogulin/bikunin precursor enhancer is included. In yet another embodiment, the ABPS (shortened version of the 100 bp distal enhancer from the α1-microglogulin/bikunin precursor [ABP] to 42 bp) enhancer is included. Such sequence is shown in SEQ ID NO: 6. In yet another embodiment, the ApoE enhancer is included. Such sequence is shown in SEQ ID NO: 7. In another embodiment, more than one enhancer is present. Such combination may include more than one copy of any of the enhancers described herein, and/or more than one type of enhancer.

In addition to a promoter, an expression cassette and/or a vector may contain other appropriate transcription initiation, termination, enhancer sequences, and efficient RNA processing signals. Such sequences include splicing and polyadenylation (polyA) signals; regulatory elements that enhance expression (i.e., WPRE (SEQ ID NO: 15); sequences that stabilize cytoplasmic mRNA; sequences that enhance translation efficiency (i.e., Kozak consensus sequence); sequences that enhance protein stability; and when desired, sequences that enhance secretion of the encoded product. In one embodiment, a polyadenylation (polyA) signal is included to mediate termination of hASS1 mRNA transcripts. Examples of other suitable poly A sequences include, e.g., bovine growth hormone (SEQ ID NO: 12), SV40, rabbit beta globin, and TK polyA, amongst others.

In one embodiment, the regulatory sequences are selected such that the total rAAV vector genome is about 2.0 to about 5.5 kilobases in size. In one embodiment, the regulatory sequences are selected such that the total rAAV vector genome is about 2.1, 2.3, 2.8, 3.1, 3.2, 3.3 or 4.0 kb in size. In one embodiment, it is desirable that the rAAV vector genome approximate the size of the native AAV genome. Thus, in one embodiment, the regulatory sequences are selected such that the total rAAV vector genome is about 4.7 kb in size. In another embodiment, the total rAAV vector genome is less about 5.2 kb in size. The size of the vector genome may be manipulated based on the size of the regulatory sequences including the promoter, enhancer, intron, poly A, etc. See, Wu et al, Mol Ther, January 2010 18 (1): 80-6, which is incorporated herein by reference.

Thus, in one embodiment, an intron is included in the vector. Suitable introns include the human beta globin IVS2 (SEQ ID NO: 13). See, Kelly et al, Nucleic Acids Research, 43 (9): 4721-32 (2015), which is incorporated herein by reference. Another suitable promoter includes the Promega chimeric intron (SEQ ID NO: 14). See, Almond, B. and Schenborn, E. T. A Comparison of pCI-neo Vector and pcDNA4/HisMax Vector. [Internet] 2000, which is incorporated herein by reference. Available from: www.promega.com/resources/pubhub/enotes/a-comparison-of-pcineo-vector-and-pcdna4hismax-vector/). Another suitable intron includes the hFIX intron (SEQ ID NO: 18). Various introns suitable herein are known in the art and include, without limitation, those found at bpg.utoledo.edu/˜afedorov/lab/eid.html, which is incorporated herein by reference. See also, Shepelev V., Fedorov A. Advances in the Exon-Intron Database. Briefings in Bioinformatics 2006, 7:178-185, which is incorporated herein by reference.

In one embodiment, the rAAV vector genome comprises a sequence selected from nt 1 to nt 3216 of SEQ ID NO: 22, nt 1 to nt 2331 of SEQ ID NO: 23, nt 1 to nt 3261 of SEQ ID NO: 24, nt 1 to nt 3325 of SEQ ID NO: 25, nt 1 to nt 2777 of SEQ ID NO: 26, nt 1 to nt 2777 of SEQ ID NO: 27, nt 1 to nt 3216 of SEQ ID NO: 28, nt 1 to nt 3066 of SEQ ID NO: 29, nt 1 to nt 2083 of SEQ ID NO: 30, nt 1 to nt 2121 of SEQ ID NO: 31, nt 1 to nt 3221 of SEQ ID NO: 32, nt 1 to nt 4040 of SEQ ID NO: 33, nt 1 to nt 2798 of SEQ ID NO: 34, or nt 1 to nt 3066 of SEQ ID NO: 35.

### 4.1.2. Compositions

In one embodiment, the rAAV.hASS1 virus is provided in a pharmaceutical composition which comprises an aqueous carrier, excipient, diluent or buffer. In one embodiment, the buffer is PBS. In a specific embodiment, the rAAV.hASS1 formulation is a suspension containing an effective amount of rAAV.hASS1 vector suspended in an aqueous solution containing 0.001% Pluronic F-68 in TMN200 (200 mM sodium chloride, 1 mM magnesium chloride, 20 mM Tris, pH 8.0). However, various suitable solutions are known including those which include one or more of: buffering saline, a surfactant, and a physiologically compatible salt or mixture of salts adjusted to an ionic strength equivalent to about 100 mM sodium chloride (NaCl) to about 250 mM sodium chloride, or a physiologically compatible salt adjusted to an equivalent ionic concentration.

For example, a suspension as provided herein may contain both NaCl and KCl. The pH may be in the range of 6.5 to 8.5, or 7 to 8.5, or 7.5 to 8. A suitable surfactant, or combination of surfactants, may be selected from among Poloxamers, i.e., nonionic triblock copolymers composed of a central hydrophobic chain of polyoxypropylene (poly(propylene oxide)) flanked by two hydrophilic chains of polyoxyethylene (poly(ethylene oxide)), SOLUTOL® HS 15 (Macrogol-15 Hydroxystearate), LABRASOL″ (Polyoxy capryllic glyceride), polyoxy 10 oleyl ether, TWEEN® (polyoxyethylene sorbitan fatty acid esters), ethanol and polyethylene glycol. In one embodiment, the formulation contains a poloxamer. These copolymers are commonly named with the letter “P” (for poloxamer) followed by three digits: the first two digits×100 give the approximate molecular mass of the polyoxypropylene core, and the last digit×10 gives the percentage polyoxyethylene content. In one embodiment Poloxamer 188 is selected. The surfactant may be present in an amount up to about 0.0005% to about 0.001% of the suspension. In another embodiment, the vector is suspended in an aqueous solution containing 180 mM sodium chloride, 10 mM sodium phosphate, 0.001% Poloxamer 188, pH 7.3.

In one embodiment, the formulation is suitable for use in human subjects and is administered intravenously. In one embodiment, the formulation is delivered via a peripheral vein by bolus injection. In one embodiment, the formulation is delivered via a peripheral vein by infusion over about 10 minutes (+5 minutes). In one embodiment, the formulation is delivered via a peripheral vein by infusion over about 20 minutes (±5 minutes). In one embodiment, the formulation is delivered via a peripheral vein by infusion over about 30 minutes (±5 minutes). In one embodiment, the formulation is delivered via a peripheral vein by infusion over about 60 minutes (±5 minutes). In one embodiment, the formulation is delivered via a peripheral vein by infusion over about 90 minutes (±10 minutes). However, this time may be adjusted as needed or desired. Any suitable method or route can be used to administer an AAV-containing composition as described herein, and optionally, to co-administer other active drugs or therapies in conjunction with the AAV-mediated delivery of hASS1 described herein. Routes of administration include, for example, systemic, oral, inhalation, intranasal, intratracheal, intraarterial, intraocular, intravenous, intramuscular, subcutaneous, intradermal, and other parental routes of administration.

In one embodiment, the formulation may contain, e.g., about 1.0×10¹¹ genome copies per kilogram of patient body weight (GC/kg) to about 1×10¹⁴ GC/kg, about 5×10¹¹ genome copies per kilogram of patient body weight (GC/kg) to about 3×10¹³ GC/kg, or about 1×10¹² to about 1×10¹⁴ GC/kg, as measured by oqPCR or digital droplet PCR (ddPCR) as described in, e.g., M. Lock et al, Hum Gene Ther Methods. 2014 April; 25 (2): 115-25. doi: 10.1089/hgtb.2013.131. Epub 2014 Feb. 14, which is incorporated herein by reference. In one embodiment, the rAAV.hASS formulation is a suspension containing at least 1×10¹³ genome copies (GC)/mL, or greater, as measured by oqPCR or digital droplet PCR (ddPCR) as described in, e.g., M. Lock et al, supra.

In order to ensure that empty capsids are removed from the dose of AAV.hASS1 that is administered to patients, empty capsids are separated from vector particles during the vector purification process, e.g., using the method discussed herein. In one embodiment, the vector particles containing packaged genomes are purified from empty capsids using the process described in U.S. Patent Appln No. 62/322,098, filed on Apr. 13, 2016, and entitled “Scalable Purification Method for AAV8”, which is incorporated by reference herein. Briefly, a two-step purification scheme is described which selectively captures and isolates the genome-containing rAAV vector particles from the clarified, concentrated supernatant of a rAAV production cell culture. The process utilizes an affinity capture method performed at a high salt concentration followed by an anion exchange resin method performed at high pH to provide rAAV vector particles which are substantially free of rAAV intermediates. Similar purification methods can be used for vectors having other capsids.

While any conventional manufacturing process can be utilized, the process described herein (and in U.S. Patent Appln No. 62/322,098) yields vector preparations wherein between 50 and 70% of the particles have a vector genome, i.e., 50 to 70% full particles. Thus for an exemplary dose of 1.6×10¹² GC/kg, and the total particle dose will be between 2.3×10¹² and 3×10¹² particles. In another embodiment, the proposed dose is one half log higher, or 5×10¹² GC/kg, and the total particle dose will be between 7.6×10¹² and 1.1×10¹³ particles. In one embodiment, the formulation is be characterized by an rAAV stock having a ratio of “empty” to “full” of 1 or less, preferably less than 0.75, more preferably, 0.5, preferably less than 0.3.

A stock or preparation of rAAV8 particles (packaged genomes) is “substantially free” of AAV empty capsids (and other intermediates) when the rAAV8 particles in the stock are at least about 75% to about 100%, at least about 80%, at least about 85%, at least about 90%, at least about 95%, or at least 99% of the rAAV8 in the stock and “empty capsids” are less than about 1%, less than about 5%, less than about 10%, less than about 15% of the rAAV8 in the stock or preparation.

Generally, methods for assaying for empty capsids and AAV vector particles with packaged genomes have been known in the art. See, e.g., Grimm et al., Gene Therapy (1999) 6:1322-1330; Sommer et al., Molec. Ther. (2003) 7:122-128. To test for denatured capsid, the methods include subjecting the treated AAV stock to SDS-polyacrylamide gel electrophoresis, consisting of any gel capable of separating the three capsid proteins, for example, a gradient gel containing 3-8% Tris-acetate in the buffer, then running the gel until sample material is separated, and blotting the gel onto nylon or nitrocellulose membranes, preferably nylon. Anti-AAV capsid antibodies are then used as the primary antibodies that bind to denatured capsid proteins, preferably an anti-AAV capsid monoclonal antibody, most preferably the BI anti-AAV-2 monoclonal antibody (Wobus et al, J. Virol. (2000) 74:9281-9293) A secondary antibody is then used, one that binds to the primary antibody and contains a means for detecting binding with the primary antibody, more preferably an anti-IgG antibody containing a detection molecule covalently bound to it, most preferably a sheep anti-mouse IgG antibody covalently linked to horseradish peroxidase. A method for detecting binding is used to semi-quantitatively determine binding between the primary and secondary antibodies, preferably a detection method capable of detecting radioactive isotope emissions, electromagnetic radiation, or colorimetric changes, most preferably a chemiluminescence detection kit. For example, for SDS-PAGE, samples from column fractions can be taken and heated in SDS-PAGE loading buffer containing reducing agent (e.g., DTT), and capsid proteins were resolved on pre-cast gradient polyacylamide gels (e.g., Novex). Silver staining may be performed using SilverXpress (Invitrogen, CA) according to the manufacturer's instructions. In one embodiment, the concentration of AAV vector genomes (vg) in column fractions can be measured by quantitative real time PCR (Q-PCR). Samples are diluted and digested with DNase I (or another suitable nuclease) to remove exogenous DNA. After inactivation of the nuclease, the samples are further diluted and amplified using primers and a TaqMan™ fluorogenic probe specific for the DNA sequence between the primers. The number of cycles required to reach a defined level of fluorescence (threshold cycle, Ct) is measured for each sample on an Applied Biosystems Prism 7700 Sequence Detection System. Plasmid DNA containing identical sequences to that contained in the AAV vector is employed to generate a standard curve in the Q-PCR reaction. The cycle threshold (Ct) values obtained from the samples are used to determine vector genome titer by normalizing it to the Ct value of the plasmid standard curve. End-point assays based on the digital PCR can also be used.

In one aspect, an optimized q-PCR method is provided herein which utilizes a broad spectrum serine protease, e.g., proteinase K (such as is commercially available from Qiagen) More particularly, the optimized qPCR genome titer assay is similar to a standard assay, except that after the DNase I digestion, samples are diluted with proteinase K buffer and treated with proteinase K followed by heat inactivation. Suitably samples are diluted with proteinase K buffer in an amount equal to the sample size. The proteinase K buffer may be concentrated to 2 fold or higher. Typically, proteinase K treatment is about 0.2 mg/mL, but may be varied from 0.1 mg/mL to about 1 mg/mL. The treatment step is generally conducted at about 55° C. for about 15 minutes, but may be performed at a lower temperature (e.g., about 37° C. to about 50° C.) over a longer time period (e.g., about 20 minutes to about 30 minutes), or a higher temperature (e.g., up to about 60° C.) for a shorter time period (e.g., about 5 to 10 minutes). Similarly, heat inactivation is generally at about 95° C. for about 15 minutes, but the temperature may be lowered (e.g., about 70 to about 90° C.) and the time extended (e.g., about 20 minutes to about 30 minutes). Samples are then diluted (e.g., 1000 fold) and subjected to TaqMan analysis as described in the standard assay.

Additionally, or alternatively, droplet digital PCR (ddPCR) may be used. For example, methods for determining single-stranded and self-complementary AAV vector genome titers by ddPCR have been described. See, e.g., M. Lock et al, Hu Gene Therapy Methods, Hum Gene Ther Methods. 2014 April; 25 (2): 115-25. doi: 10.1089/hgtb.2013.131. Epub 2014 Feb. 14.

### 4.2 Patient Population

As discussed above, a subject having CTLN1 of any severity is the intended recipient of the compositions and methods described herein.

Subjects may be permitted to continue their standard of care treatment(s) (e.g., protein restricted diet, and/or medications (including nitrogen scavenger therapy and carnitine)) prior to and concurrently with the gene therapy treatment at the discretion of their caring physician. In the alternative, the physician may prefer to stop standard of care therapies prior to administering the gene therapy treatment and, optionally, resume standard of care treatments as a co-therapy after administration of the gene therapy.

Desirable endpoints of the gene therapy regimen are an increase in ASS activity resulting in citrulline levels below about 100 μmol/L and/or ammonia levels below about 100 μmol/L. In another embodiment, any reduction in citrulline, glutamine, and/or ammonia levels is desirable. Other suitable clinical outcomes may include reduction in the use of scavenger, less restrictive diet or no need for liver transplant. In one embodiment, patients achieve reduced circulating ASS1 levels after treatment with rAAV.hASS1, alone and/or combined with the use of adjunctive treatments.

### 4.3. Dosing & Route of Administration

In one embodiment, the rAAV.hASS1 vector is delivered as a single dose per patient. In one embodiment, the subject is delivered the minimal effective dose (MED) (as determined by preclinical study described in the Examples herein). As used herein, MED refers to the rAAV.hASS1 dose required to achieve ASS1 activity resulting in citrulline levels below about 100 μmol/L and/or ammonia levels below about 100 μmol/L.

As is conventional, the vector titer is determined on the basis of the DNA content of the vector preparation. In one embodiment, quantitative PCR or optimized quantitative PCR as described in the Examples is used to determine the DNA content of the rAAV.hASS1 vector preparations. In one embodiment, digital droplet PCR as described in the Examples is used to determine the DNA content of the rAAV.hASS1 vector preparations. In one embodiment, the dosage is about 1×10¹¹ genome copies (GC)/kg body weight to about 1×10¹³ GC/kg, inclusive of endpoints. In one embodiment, the dosage is 5×10¹¹ GC/kg. In another embodiment, the dosage is 5×10¹² GC/kg. In specific embodiments, the dose of rAAV.hASS1 administered to a patient is at least 5×10¹¹ GC/kg, 1×10¹² GC/kg, 1.5×10¹² GC/kg, 2.0×10¹² GC/kg, 2.5×10¹² GC/kg, 3.0×10¹² GC/kg, 3.5×10¹² GC/kg, 4.0×10¹² GC/kg, 4.5×10¹² GC/kg, 5.0×10¹² GC/kg, 5.5×10¹² GC/kg, 6.0×10¹² GC/kg, 6.5×10¹² GC/kg, 7.0×10¹² GC/kg, or 7.5×10¹² GC/kg. Also, the replication-defective virus compositions can be formulated in dosage units to contain an amount of replication-defective virus that is in the range of about 1.0×10⁹ GC to about 1.0×10¹⁵ GC. As used herein, the term “dosage” can refer to the total dosage delivered to the subject in the course of treatment, or the amount delivered in a single (of multiple) administration.

In some embodiments, rAAV.hASS1 is administered in combination with one or more therapies for the treatment of CTLN1, such as a low protein diet or administration nitrogen scavenger therapy or dialysis.

### 4.4. Measuring Clinical Objectives

Measurements of efficacy of treatment can be measured by transgene expression and activity as determined by ammonia or citrulline levels and/or ASS1 activity. Further assessment of efficacy can be determined by clinical assessment of dietary citrulline tolerance.

As used herein, the rAAV.hASS1 vector herein “functionally replaces” or “functionally supplements” the patients defective ASS1 with active ASS1 when the patient expresses a sufficient level of ASS1 to achieve ASS1 activity resulting in citrulline and/or ammonia levels less than about 100 μmol/L. In another embodiment, the rAAV.hASS1 vector functionally replaces or functionally supplements the patient's defective ASS1 when partial rescue is provided. This allows for treatment with a combination of scavengers and dietary control. In one embodiment, the treatment provides sufficient rescue such that liver transplantation is not required. In another embodiment, a reduction in the rate of complications such as viral diseases (e.g., flu) is desired.

### 5. EXAMPLES

The following examples are illustrative only and are not intended to limit the present invention.

### Example 1: AAV Vectors Containing hASS1

An exemplary gene therapy vector AAV8.TBG.PI.hASS1co. WPRE.bGH was constructed by an AAV8 vector bearing a codon-optimized human ASS1 cDNA (hASS1co) under the control of TBG, a hybrid promoter based on the human thyroid hormone-binding globulin promoter and microglobin/bikunin enhancer. The ASS1 expression cassette was flanked by AAV2 derived inverted terminal repeats (ITRs) and the expression was driven by a hybrid of the TBG enhancer/promoter and the Woodchuck Hepatitis Virus (WHP) posttranscriptional regulatory element (WPRE) as an enhancer. The transgene also included the Promega SV40 misc intron (PI) and a bovine growth hormone polyadenylation signal (bGH).

### Another Exemplary Gene Therapy Vector

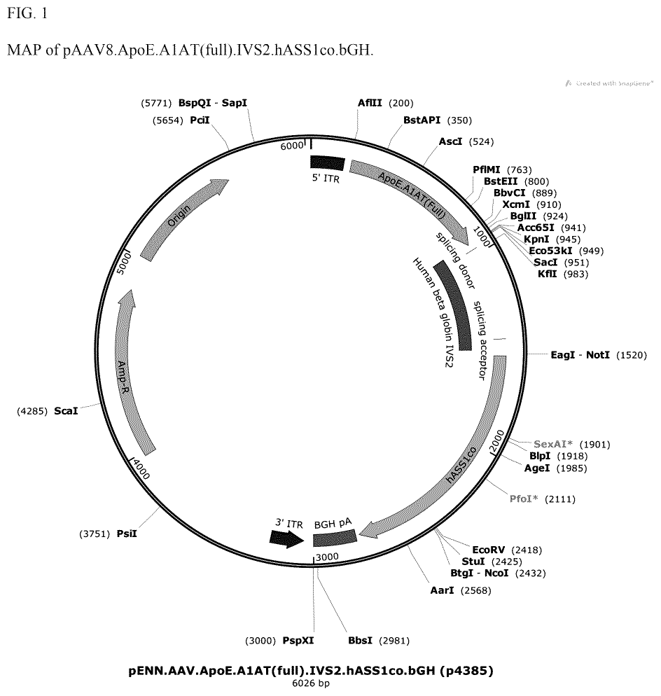

AAV8.ApoE.A1AT (full).IVS2.hASS1co.bGH was constructed by an AAV8 vector bearing a codon-optimized human ASS1 cDNA (hASS1co) under the control of A1AT promoter and a ApoE enhancer (FIG. 1). The ASS1 expression cassette was flanked by AAV2 derived inverted terminal repeats (ITRs) and the expression was driven by the ApoE.A1AT enhancer/promoter. The transgene also included the human beta globin IVS2 as an intron and a bovine growth hormone polyadenylation signal (bGH).

The vector AAV8.TBG.PI.hASS1co.bGH was constructed as described above without WPRE as an enhancer.

The AAV8.LSP.IVS2.hASS1co.bGH vector encodes a codon-optimized human ASS1 cDNA (hASS1co) under the control of a liver-specific promoter (LSP), with intervening sequence 2 (IVS2) and a bovine growth hormone polyadenylation signal (bGH).

The vector was prepared using conventional triple transfection techniques in 293 cells as described e.g., by Mizukami, Hiroaki, et al. A Protocol for AAV vector production and purification. Diss. Di-vision of Genetic Therapeutics, Center for Molecular Medicine, 1998., which is incorporated herein by reference. All vectors were produced by the Vector Core at the University of Pennsylvania as previously described [Lock, M., et al, Hum Gene Ther, 21:1259-1271 (2010)].

### Example 2: Natural History Study

All animal procedures were performed in accordance with protocols approved by the Institutional Animal Care and Use Committee (IACUC) of the University of Pennsylvania.

Citrullinemia is an autosomal recessive disease caused by mutations in argininosuccinate synthase (ASS1) enzyme that catalyzes the synthesis of argininosuccinate from citrulline and aspartate, results in citrullinemia and buildup of ammonia. ASS1^{fold/fold} mouse express deficient argininosuccinate synthase 1 (ASS1) [Harris B S, et al., Follicular dystrophy: a new skin and hair mutation on mouse Chromosome 2. MGI Direct Data Submission. 2007]. FOLD allele mice have a T389I substitution in exon 15 leading to an unstable protein structure with normal ASS1 mRNA and protein levels. Homozygotes survive up to 3 weeks or longer, have 5-10% enzyme activity and display clinical and biochemical parameters similar to CTLN1. At 1 week of age fold homozygotes lack hair such that they can be distinguished from their control littermates, and at 2 weeks of age fold homozygotes have wrinkled skin, a sparse coat does grow in. By P14 mice show a 10- to 40-fold increase in the levels of citrulline, and a 1.5- to threefold increase in the plasma levels of many amino acids, including glutamine, cystine, methionine, and lysine and arginine, glutamic acid, leucine, and ornithine levels are decreased.

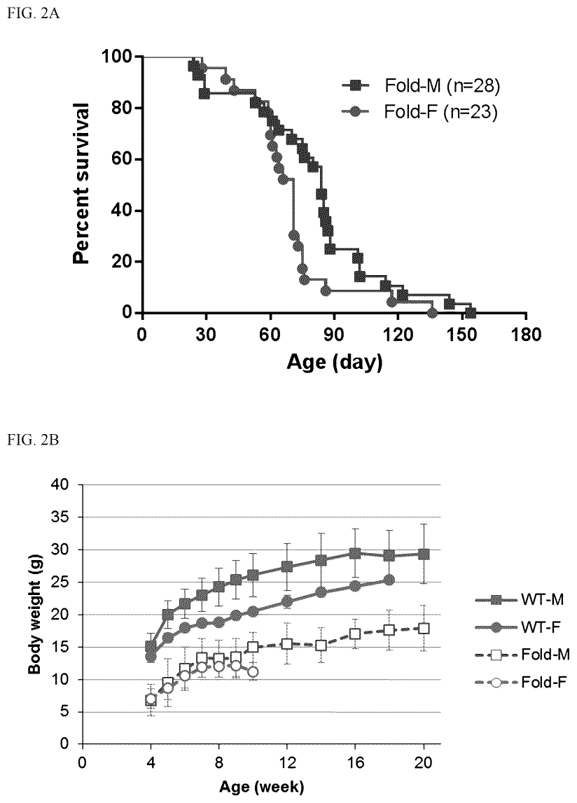

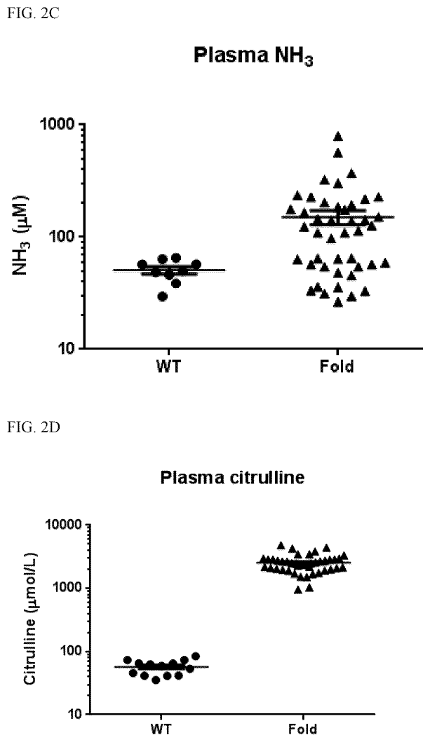

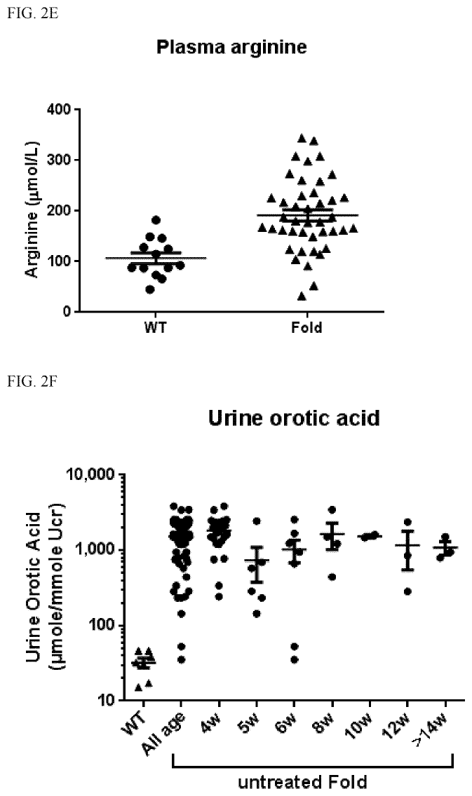

ASS1^{fold/fold} mice thus served as a mouse model for Citrullinemia. Survival curve was generated based on the observation of 28 male and 23 female ASS1^{fold/fold} mice (FIG. 2A). The result demonstrated that functionally deficient ASS1 reduced the lifespan of ASS1^{fold/fold} mice significantly.

As a second parameter for normal development and growth, the body weights of ASS1^{fold/fold} mice and healthy littermates after weaning were closely monitored and recorded. The results showed that female ASS1^{fold/fold} mice weight remained relatively constant and the body weights of male ASS1^{fold/fold} mice reached plateau at about 8 weeks old while both genders of wild-type littermates exhibited a steady growth over the observation period (FIG. 2B). It further demonstrated that functionally deficient ASS1 compromised the development and growth of the mice.

Additional testing was done on the untreated ASS1^{fold/fold} mice and healthy littermates, including measurement of plasma ammonia (FIG. 2C), plasma citrulline (FIG. 2D). plasma arginine (FIG. 2E), urine orotic acid (FIG. 2E). Ammonia, citrulline, arginine and orotic acid levels were all elevated in untreated ASS1^{fold/fold} mice as compared to healthy littermates.

### Example 3: AAV8.hASS1 Vectors in the Model of Citrullenemia

To evaluate the efficacy and determine the dose-dependent effects of AAV8.hASS1co vectors, ASS1^{fold/fold} mice were injected with 1×10¹¹ GC/mouse or 3×10¹¹ GC/mouse of the gene therapy vectors intravenously at birth, as shown in the Table 1 below. Wild-type and heterozygous littermates served as controls.

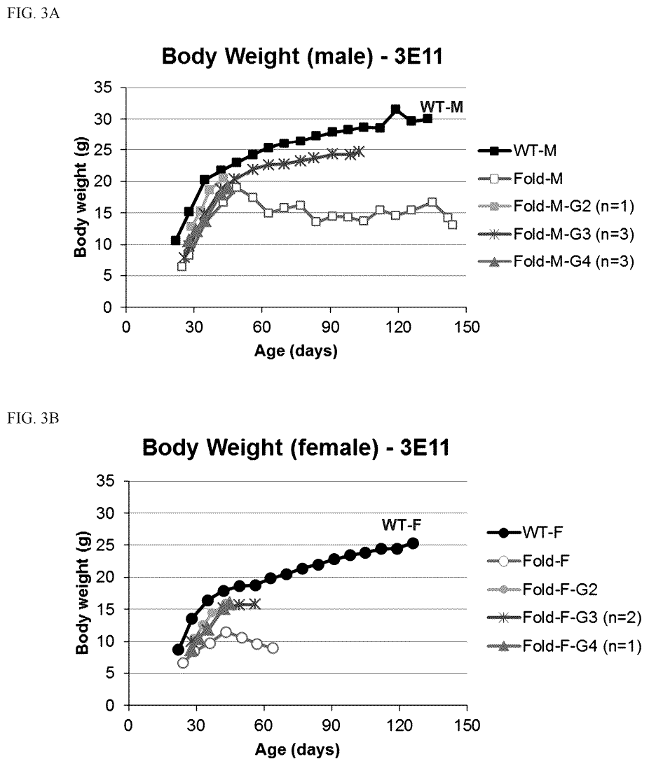

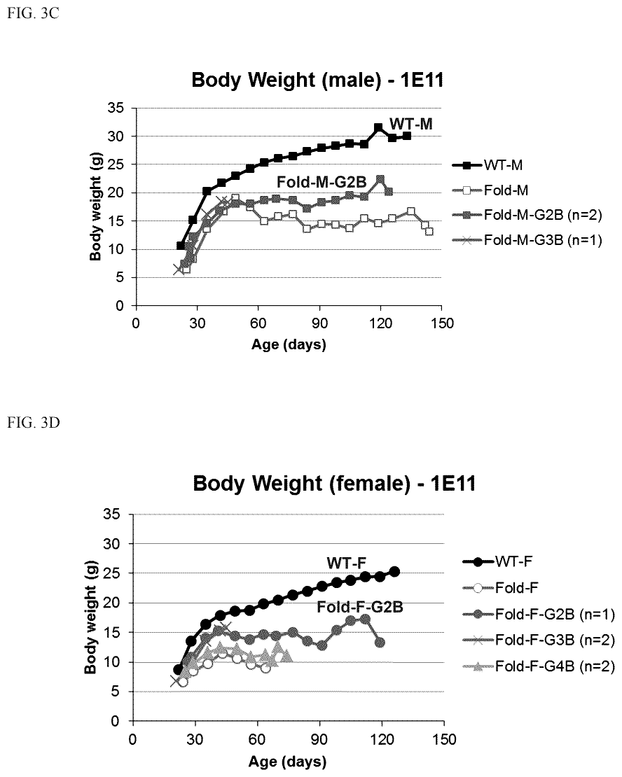

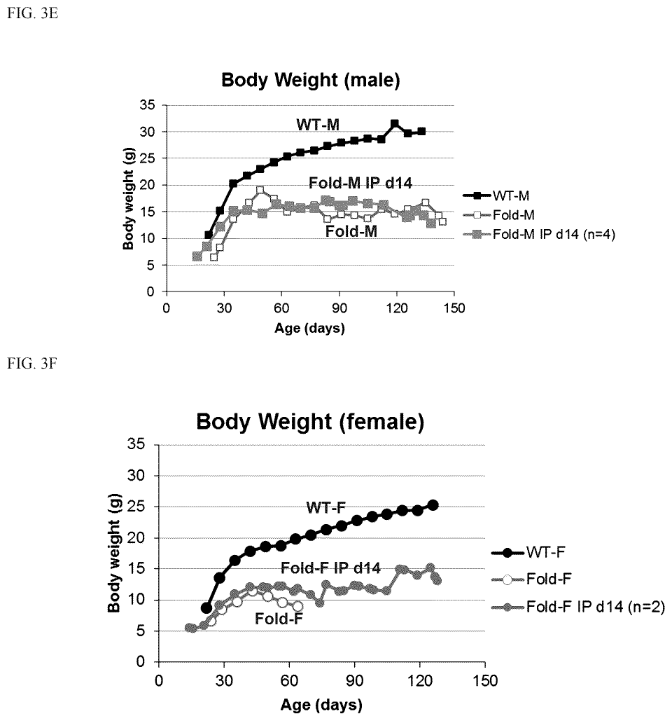

3×10¹¹ GC/pup of AAV8.LSP.IVS2.hASS1co.bGH vector successfully increased the rate of weight gain in male ASS1^{fold/fold} mice (FIG. 3A). In females, 3×10¹¹ GC/pup of all three tested vectors rescued the reduction in body weight upon growth (FIG. 3B). Meanwhile, mice injected intravenously at birth with 1×10¹¹ GC/pup of the vectors demonstrated a slight increase in weights (FIG. 3C and FIG. 3D). Mice which received an intraperitoneal injection of the AAV8.TBG.PI.hASS1co. WPRE.bGH vector at postnatal day 14 did not exhibit any increase in body weight (FIG. 3E and FIG. 3F).

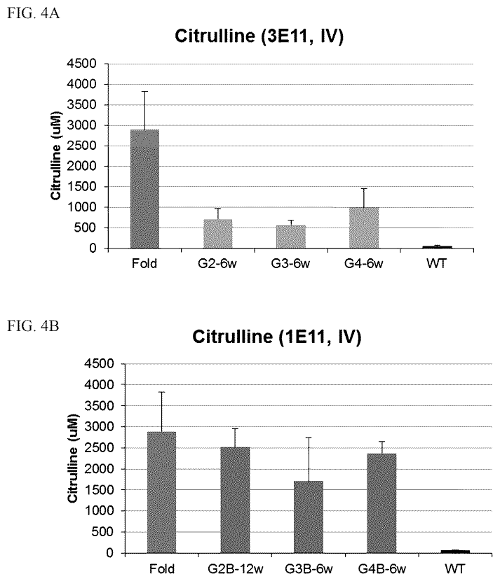

A further experiment was performed to assess the survival of citrullenemia mice treated with the AAV8.hAASco vectors. To assess citrulline accumulation in the blood, concentration of citrulline in ASS1^{fold/fold} mice with intravenous injections of AAV8.TBG.PI.hASS1co.WPRE.bGH, AAV8.LSP.IVS2.hASS1co.bGH or AAV8.TBG.PI.hASS1co.bGH on postnatal day 0 at 1×10¹¹ or 3×10¹¹ GC/pup were examined (FIG. 4B and FIG. 4A, respectively). Injection of 1×10¹¹ GC/pup of the vectors resulted in a minor decrease in citrulline compared to the untreated ASS1^{fold/fold} mice (FIG. 4B), while 3×10¹¹ GC/pup of the vectors successfully brought the citrulline level down (FIG. 4A).

A further study of expression and enzyme activity of ASS1 in the injected ASS1^{fold/fold} mice is performed. Livers from the tested mice injected with AAV8.hASSco vectors and the healthy littermate controls are collected and lysates are prepared. The mRNA is extracted and the expression of ASS1 is evaluated via RT-PCR. The protein expression of ASS1 is determined by western blot and immunohistochemistry. Experiments are also performed to assess the ASS1 activity in the ASS1^{fold/fold} mice treated with the vector as well as controls.

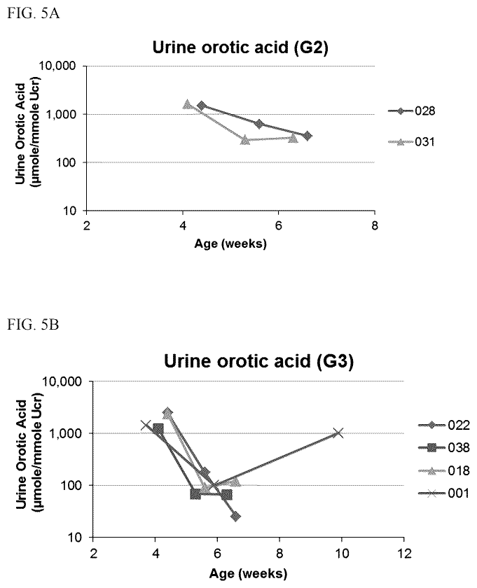

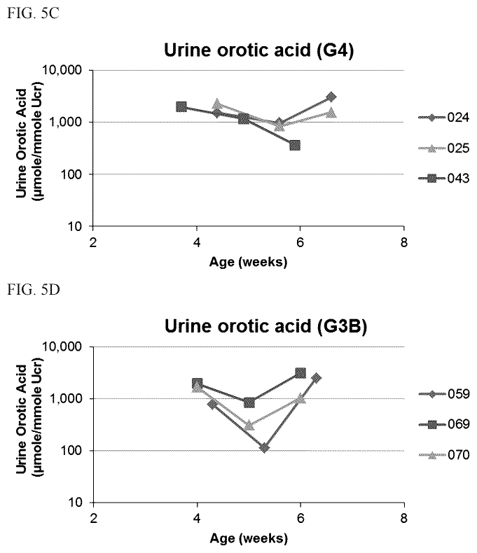

An additional experiment was performed, where weight (FIG. 5A), ALT (FIG. 5B), and alkaline phosphatase (FIG. 5C) were measured on ASS1^{fold/fold} mice treated with 1×10¹¹ GC/pup of the vector AAV8.TBG.PI.hASS1co. WPRE.bGH. Wild-type and heterozygous littermates serves as controls.

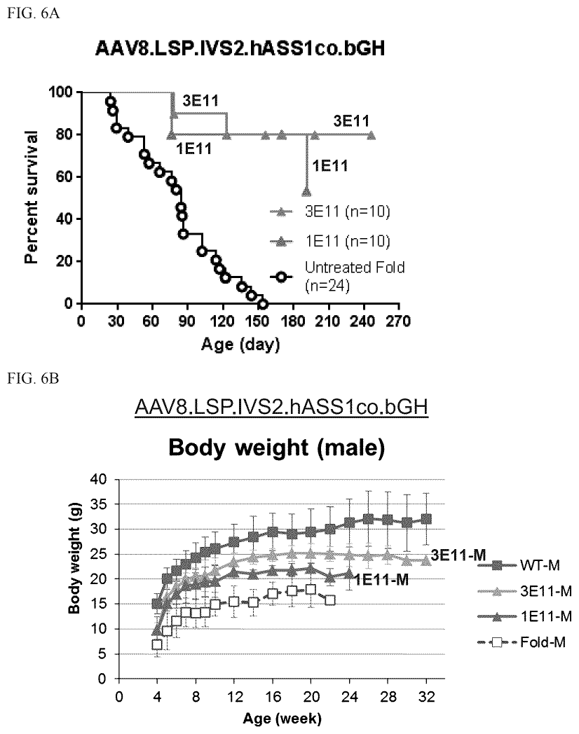

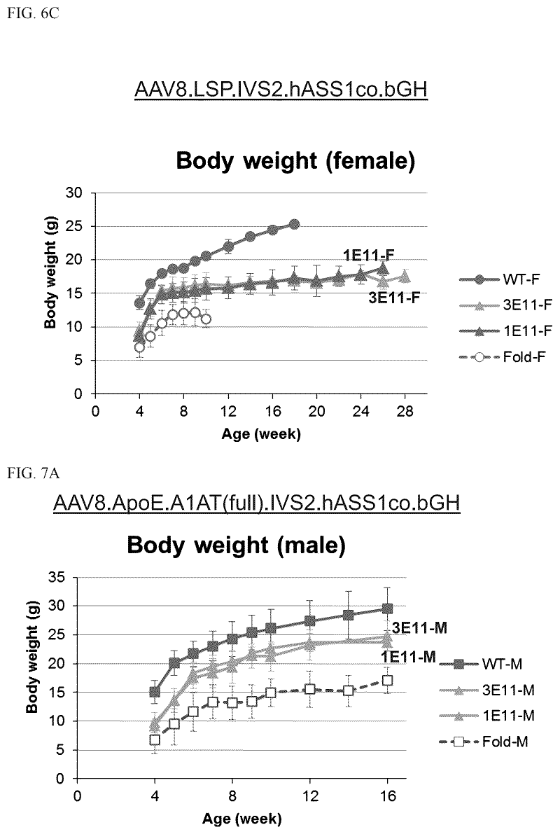

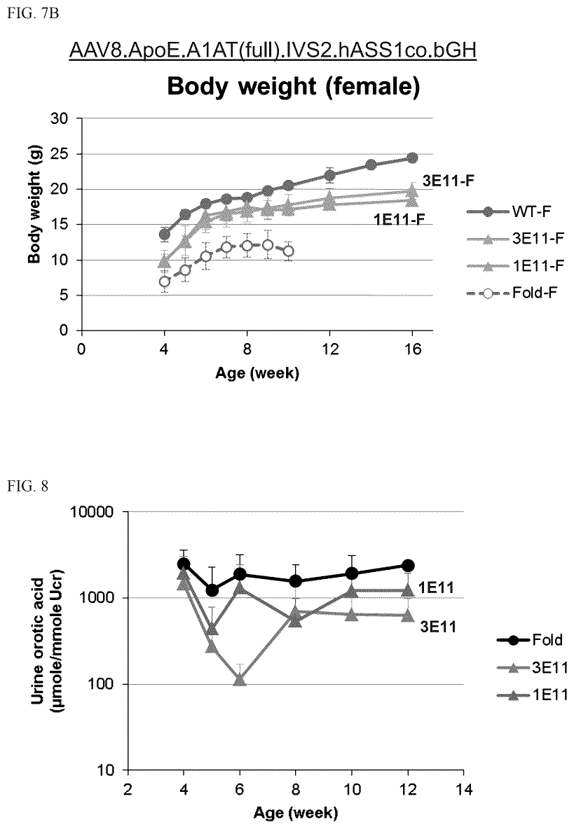

Urine orotic acid levels were measured in ASS1^{fold/fold} mice injected intravenously at birth with 3×10¹¹ GC/pup of AAV8.TBG.PI.hASS1co.WPRE.bGH (FIG. 6A), AAV8.LSP.IVS2.hASS1co.bGH (FIG. 6B), AAV8.TBG.PI.hASS1co.bGH (FIG. 6C), or with 1×10¹¹ GC/pup of AAV8.LSP.IVS2.hASS1co.bGH (FIG. 6D).

### Example 4: Additional Vectors

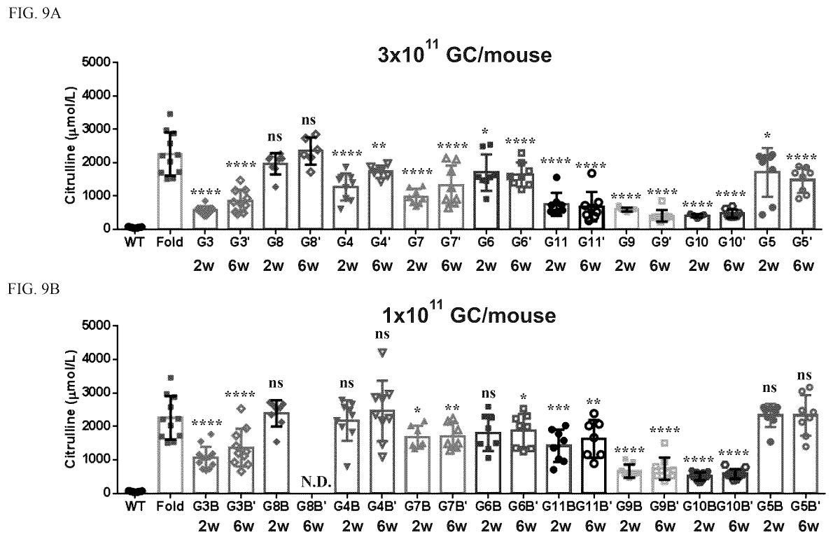

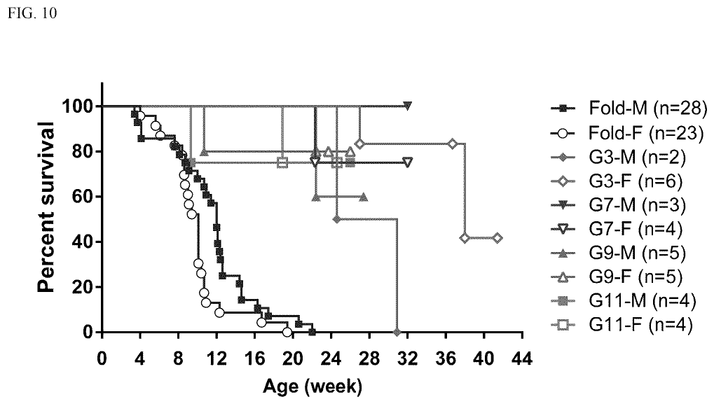

Additional AAV vectors as shown in Table below were produced. ASS1^{fold/fold} mice were injected on postnatal day 0 at 1×10¹¹ or 3×10¹¹ GC/pup with the noted vectors. Citrulline levels at two and six week post injection were measured (FIGS. 9A and 9B).

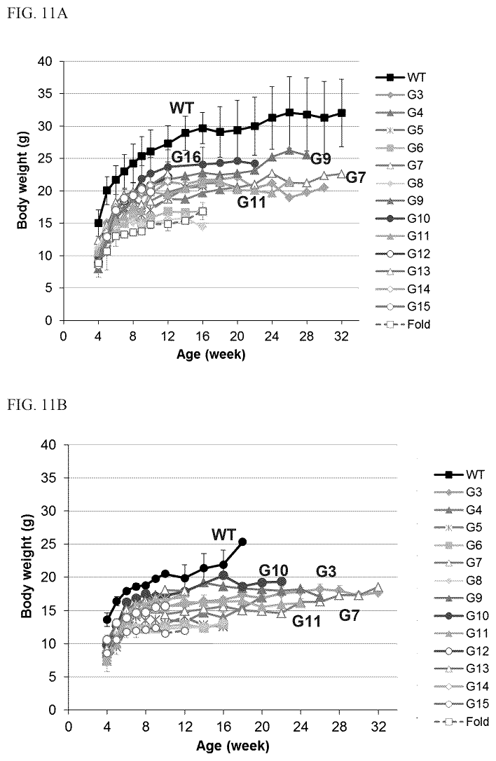

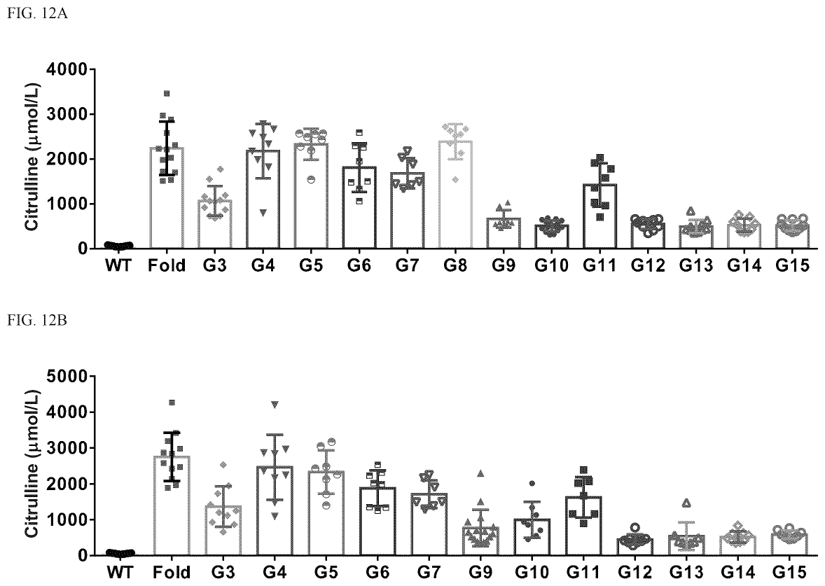

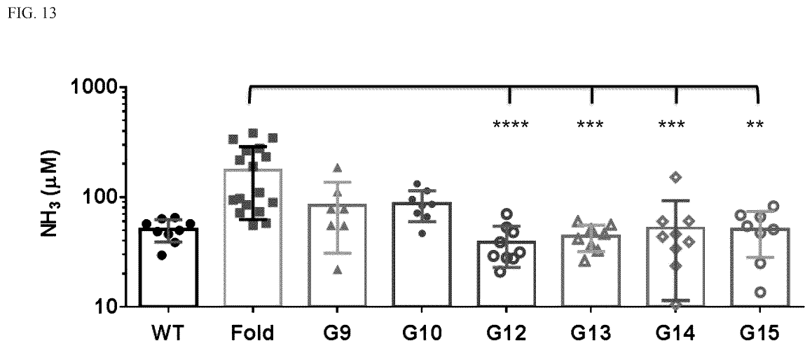

FIG. 10 shows a survival curve of male and female mice injected with G3, G7, G9 and G11. All vectors tested provided significant increase in survival of ASS1^{fold/fold} mice. Likewise, all vectors tested provided an increase in body weight in both male (FIG. 11A) and female (FIG. 11B) mice, a decrease in citrulline levels at 2 weeks (FIG. 12A) and 6 weeks (FIG. 12B) post injection, and a decrease in ammonia levels (FIG. 13). In conclusion, a single injection of AAV8.hASSco vectors resulted in substantial blood citrulline reduction and concomitant functional correction when administered intravenously in ASS1-deficient mice.

### Example 5: Long-Term Rescue of a Hypomorphic Lethal Murine Model of Citrullinemia Type I by Liver-Directed, AAV8-Mediated Gene Therapy

Citrullinemia type I (CTLN1) is an autosomal, recessive disorder of the urea cycle caused by a deficiency of argininosuccinate synthase 1 (ASS1). The clinical spectrum of CTLN1 ranges from a severe neonatal onset form to a milder form with later onset. Affected patients have persistent elevated plasma citrulline levels and are at risk of life-threatening elevation of ammonia that can lead to irreversible cognitive impairment, coma, and death. Current treatment for CTLN1 patients, which includes a low protein diet, supplementation of arginine and administration of nitrogen scavengers, is often unable to prevent ongoing hyperammonemic crises. Liver transplantation has shown successful reduction of plasma ammonia and citrulline levels, but donor liver is limiting, the procedure itself carries significant morbidity, and immunosuppressive drugs are necessary for the duration of the graft. Therefore, there is a need for other approaches to therapy for CTLN1.

TABLE 1
Summary of Vectors for liver directed therapy for treatment of citrullinemia

| | | | | | Key elements in vector | | |
|---|---|---|---|---|---|---|---|
| | | | | | Enhancer- | | |
| Grp | Vector | | | | Promoter | Intron | Transgene |
| G2 | AAV8.TBG.PI.hASS1co.WPRE.bGH (p3795) | | | | ABPx2- | PI | hASS1co |
| | | | | | TBG | | |
| G3 | AAV8.TBG.IVS2.hASS1co.bGH (p4169) | | | | ABPx2- | IVS2 | hASS1co |
| | | | | | TBG | | |
| G4 | AAV8.TBG.PI.hASS1co.bGH (p4157) | | | | ABPx2- | PI | hASS1co |
| | | | | | TBG | | |
| G5 | AAV8.EnTTR.TTR.hASS1co.bGH (p4319) | | | | EnTTR- | — | hASS1co |
| | | | | | TTR | | |
| G6 | AAV8.En34.A1AT.hASS1co.bGH (p4320) | | | | En34-A1AT | — | hASS1co |
| G7 | AAV8.TBG.PI.hASS1-native.bGH (p4339) | | | | ABPx2- | PI | hASS1- |
| | | | | | TBG | | native |
| G8 | AAV8.TBG.hFIXintron.hASS1co.bGH (p4382) | | | | ABPx2- | hFIX | hASS1co |
| | | | | | TBG | intron | |
| G9 | AAV8.ApoE.A1AT.IVS2.hASS1co.bGH (p4383) | | | | ApoE- | IVS2 | hASS1co |
| | | | | | A1AT | | |
| G10 | AAV8.ApoE.A1AT(full).IVS2.hASS1co.bGH | | | | ApoE- | IVS2 | hASS1co |
| | (p4385) | | | | A1AT (full) | | |
| G11 | AAV8.En34.A1AT.PI.hASS1co.bGH (p4340) | | | | En34-A1AT | PI | hASS1co |
| G12 | AAV8.ApoE.A1AT(full).IVS2.hASS1- | | | | ApoE- | IVS2 | hASS1- |
| | native.bGH (p4456) | | | | A1AT (full) | | native |
| G13 | AAV8.ApoE.A1AT.IVS2.hASS1-native.bGH | | | | ApoE- | IVS2 | hASS1- |
| | (p4457) | | | | A1AT | | native |
| G14 | AAV8.TBG.IVS2.hASS1-native.bGH (p4458) | | | | ABPx2- | IVS2 | hASS1- |
| | | | | | TBG | | native |
| G15 | AAV8.EnTTR.TTR.IVS2.hASS1-native.bGH | | | | EnTTR- | IVS2 | hASS1- |
| | (p4459) | | | | TTR | | native |
| | | | | w2 citrulline (μM) | | w6 citrulline | |
| | | | | Mean ± STD | | (% reduction) | |
| | | Size (bp) | Yield (GC)/ | 3E11 | 1E11 | 3E11 | 1E11 |
| | Grp | (ITR-ITR) | Cellstack | GC/mouse | GC/mouse | GC/mouse | GC/mouse |
| | G2 | 3325 | 1.21E+14 | 714 ± 161 | N.D. | 68.4% | N.D. |
| | | | | (n = 5)**** | | | |
| | G3 | 3261 | 8.60E+13 | 564 ± 122 | 1065 ± 329 | 75 | 52 |
| | | | | (n = 13)**** | (n = 12) | | |
| | G4 | 2777 | 1.26E+14 | 1272 ± 406 | 2176 ± 607 | 43 | 3 |
| | | | | (n = 9) | (n = 9) | | |
| | G5 | 2121 | 9.90E+13 | 1707 ± 732 | 2328 ± 348 | 24 | −4 |
| | | | | (n = 8) | (n = 8) | | |
| | G6 | 2083 | 6.60E+13 | 1702 ± 549 | 1808 ± 542 | 24 | 19 |
| | | | | (n = 8) | (n = 8) | | |
| | G7 | 2777 | 1.17E+14 | 980 ± 231 | 1681 ± 337 | 56 | 25 |
| | | | | (n = 8) | (n = 7) | | |
| | G8 | 4040 | 1.38E+14 | 1966 ± 319 | 2389 ± 392 | 12 | −7 |
| | | | | (n = 8) | (n = 8) | | |
| | G9 | 3066 | 6.75E+13 | 591 ± 57 | 664 ± 196 | 74 | 70 |
| | | | | (n = 9)**** | (n = 9)** | | |
| | G10 | 3216 | 1.01E+14 | 404 ± 45 | 509 ± 118 | 82 | 77 |
| | | | | (n = 9)**** | (n = 16)**** | | |
| | G11 | 2331 | 1.17E+14 | 746 ± 348 | 1420 ± 485 | 67 | 37 |
| | | | | (n = 8)** | (n = 8) | | |
| | G12 | 3216 | 7.27E+13 | N.D. | 548 ± 111 | N.D. | 76 |
| | | | | | (n = 10)**** | | |
| | G13 | 3066 | 1.10E+14 | N.D. | 494 ± 148 | N.D. | 78 |
| | | | | | (n = 10)**** | | |
| | G14 | 3221 | 7.12E+13 | N.D. | 523 ± 147 | N.D. | 77 |
| | | | | | (n = 9)**** | | |
| | G15 | 2798 | 1.37E+14 | N.D. | 513 ± 119 | N.D. | 77 |
| | | | | | (n = 10)**** | | |

| | |
|---|---|
| Fold | 2239 ± 596 (n = 13) |

AAV vector-based gene therapy provides an alternative to current treatment options as long as the vector delivers sufficient and sustained transgene expression in the liver without substantial toxicity. Several candidate AAV8 vectors for CTLN1 were generated with different liver-specific promoters, introns, and cDNA sequences (native or codon-optimized hASS1 cDNA). In vivo evaluation of vectors was performed in a murine model of CTLN1 (ASS1^{fold/fold}). Homozygous ASS1^{fold/fold} (fold) mice carried a hypomorphic mutation and display lethality after weaning. Half of the untreated fold mice perished before the age of 12 weeks old, while a few (5%) lived up to 5 months. In addition to significantly elevated plasma citrulline levels, untreated fold mice had significantly reduced body weight, variable elevated plasma ammonia levels and urine orotic acid levels, and they were not fertile.

Four-week-old fold mice were dosed via retro-orbital or intraperitoneal injection with 3×10¹¹ GC or 1×10¹¹ GC of vector. Reduction of plasma citrulline levels was chosen as the main criteria to differentiate the performance of different vectors. A lead vector containing the ApoE enhancer-alpha 1 antitrypsin promoter and the beta globulin intervening sequence 2 achieved 77% reduction of citrulline levels two weeks post vector administration at the dose of 1×10¹¹ GC. Intron played an important role in expression of ASS1 and vectors carrying the same promoter with other introns, or no intron, showed significantly perturbed efficiency in reducing citrulline levels. Vector with native cDNA sequences performed slightly better than a vector with codon-optimized cDNA sequences. Fold mice treated with the top candidate vectors gained weight, became fertile, and survived more than 9 months (still on-going).

### (Sequence Listing Free Text)

The following information is provided for sequences containing free text under numeric identifier <223>.

| | Amino Acid | | |
|---|---|---|---|
| SEQ | or Nucleic | | |
| ID NO | Acid Sequence | Free text under <223> | Description |
| 1 | Amino Acid | | human ASS1 |
| 2 | Nucleic Acid | <223> constructed sequence | engineered cDNA for human ASS1 |
| 3 | Nucleic Acid | | cDNA for native human ASS1 |
| 4 | Nucleic Acid | <223> constructed sequence | En34 enhancer |
| 5 | Nucleic Acid | <223> constructed sequence | EnTTR enhancer |
| 6 | Nucleic Acid | <223> constructed sequence | ABPS enhancer |
| 7 | Nucleic Acid | <223> constructed sequence | ApoE enhancer |
| 8 | Nucleic Acid | <223> constructed sequence | TBG-S1 |
| 9 | Nucleic Acid | <223> constructed sequence | TBG promoter |
| 10 | Nucleic Acid | <223> constructed sequence | A1AT promoter |
| 11 | Nucleic Acid | <223> constructed sequence | TTR promoter |
| 12 | Nucleic Acid | <223> constructed sequence | bGH polyA |
| 13 | Nucleic Acid | <223> constructed sequence | human beta globin IVS2 |
| 14 | Nucleic Acid | <223> constructed sequence | Promega ® chimeric intron |
| 15 | Nucleic Acid | <223> constructed sequence | WPRE |
| 16 | Nucleic Acid | <223> constructed sequence | 5′ ITR |
| 17 | Nucleic Acid | <223> constructed sequence | 3′ ITR |
| 18 | Nucleic Acid | <223> constructed sequence | hFIX intron |
| 19 | Amino Acid | <223> AAV8 | AAV8 capsid |
| 20 | Nucleic Acid | <223> constructed sequence | A1AT promoter combined with an ApoE |
| | | | enhancer |
| 21 | Nucleic Acid | <223> constructed sequence | liver-specific promoter LSP |
| 22 | Nucleic Acid | <223> constructed sequence | ApoE.A1AT(full).IVS2.hASS1co.bGH cis |
| | | | plasmid |
| 23 | Nucleic Acid | <223> constructed sequence | En34.A1AT.PI.hASS1co.bGH cis plasmid |
| 24 | Nucleic Acid | <223> constructed sequence | TBG.IVS2.hASS1co.bGH cis plasmid |
| 25 | Nucleic Acid | <223> constructed sequence | TBG.PI.hASS1co.WPRE.bGH cis plasmid |
| 26 | Nucleic Acid | <223> constructed sequence | TBG.PI.hASS1co.bGH cis plasmid |
| 27 | Nucleic Acid | <223> constructed sequence | TBG.PI.hASS1-native.bGH cis plasmid |
| 28 | Nucleic Acid | <223> constructed sequence | ApoE.A1AT(full).IVS2.hASS1-native.bGH |
| | | | cis plasmid |
| 29 | Nucleic Acid | <223> constructed sequence | ApoE.A1AT.IVS2.hASS1co.bGH cis plasmid |
| 30 | Nucleic Acid | <223> constructed sequence | En34.A1AT.hASS1co.bGH cis plasmid |
| 31 | Nucleic Acid | <223> constructed sequence | EnTTR.TTR.hASS1co.bGH cis plasmid |
| 32 | Nucleic Acid | <223> constructed sequence | TBG.IVS2.hASS1-native.bGH cis plasmid |
| 33 | Nucleic Acid | <223> constructed sequence | TBG.hFIXintron.hASS1co.bGH cis plasmid |
| 34 | Nucleic Acid | <223> constructed sequence | EnTTR.TTR.IVS2.hASS1-native.bGH cis |
| | | | plasmid |
| 35 | Nucleic Acid | <223> constructed sequence | ApoE.A1AT.IVS2.hASS1-native.bGH cis |
| | | | plasmid |
| 36 | Nucleic Acid | | AAV8 capsid |

All publications cited in this specification are incorporated herein by reference in their entireties, as are U.S. Provisional Patent Application No. 62/453,424, filed Feb. 1, 2017, and U.S. provisional Patent Application No. 62/469,650, filed Mar. 10, 2017. Similarly, the SEQ ID NOs which are referenced herein and which appear in the appended Sequence Listing are incorporated by reference. While the invention has been described with reference to particular embodiments, it will be appreciated that modifications can be made without departing from the spirit of the invention. Such modifications are intended to fall within the scope of the appended claims.

1. A recombinant adeno-associated virus (rAAV) useful as a liver-directed therapeutic for citrullinemia, said rAAV comprising an AAV capsid, and a vector genome packaged therein, said vector genome comprising:
(a) an AAV 5′ inverted terminal repeat (ITR) sequence;
(b) a promoter,
(c) a coding optimized sequence encoding a human Argininosuccinate Synthase 1 (ASS1);
(d) an AAV 3′ ITR.


SEQUENCE LISTING (36 sequences)

SEQ ID NO: 1 - protein, 412 aa, Homo sapiens
MSSKGSVVLAYSGGLDTSCILVWLKEQGYDVIAYLANIGQKEDFEEARKKALKLGAKKVFIEDVSREFVEEFIWPAIQSSALYEDRYLLGTSLARPCIARKQVEIAQREGAKYVSHGATGKGNDQVRFELSCYSLAPQIKVIAPWRMPEFYNRFKGRNDLMEYAKQHGIPIPVTPKNPWSMDENLMHISYEAGILENPKNQAPPGLYTKTQDPAKAPNTPDILEIEFKKGVPVKVTNVKDGTTHQTSLELFMYLNEVAGKHGVGRIDIVENRFIGMKSRGIYETPAGTILYHAHLDIEAFTMDREVRKIKQGLGLKFAELVYTGFWHSPECEFVRHCIAKSQERVEGKVQVSVLKGQVYILGRESPLSLYNEELVSMNVQGDYEPTDATGFININSLRLKEYHRLQSKVTAK

SEQ ID NO: 2 - DNA, 1236 bp, synthetic construct
  misc_feature 1..1236: note = constructed sequence
  source 1..1236: mol_type = other DNA
atgagcagcaagggctctgtggtgctggcctactctggcggcctggacaccagctgtatcctcgtgtggctgaaagaacagggctacgacgtgatcgcctacctggccaacatcggccagaaagaggacttcgaggaagcccggaagaaggccctgaagctgggcgccaagaaggtgttcatcgaggacgtgtcccgcgagttcgtggaagagttcatctggcccgccatccagagcagcgccctgtacgaggacagatacctgctgggcaccagcctggccagaccctgtatcgcccggaaacaggtggaaatcgcccagcgcgagggcgccaaatacgtgtctcacggcgccaccggcaagggcaacgaccaggtgcgctttgagctgagctgctactccctggccccccagatcaaagtgatcgccccttggcggatgcccgagttctacaaccggttcaagggccggaacgacctgatggaatacgccaagcagcacggcatccccatccccgtgacccccaagaacccttggagcatggacgagaacctgatgcacatcagctacgaggccggcatcctggaaaaccccaagaatcaggcccctcccggcctgtacacaaagacccaggaccctgccaaggcccccaacacccccgacattctggaaatcgagttcaagaaaggcgtgcccgtgaaagtgaccaacgtgaaggacggcaccacccaccagacctccctggaactgttcatgtacctgaacgaggtggccggcaagcacggcgtgggcagaatcgacatcgtggaaaacagattcatcggcatgaagtcccggggcatctacgagacaccagccggcaccatcctgtaccacgcccacctggatatcgaggccttcaccatggaccgggaagtgcggaagatcaagcagggcctgggcctgaagttcgccgagctggtgtacacaggcttttggcacagccccgagtgcgagtttgtgcggcactgcattgccaagagccaggaacgggtggaaggcaaggtgcaggtgtccgtgctgaagggccaggtgtacattctgggcagagagagccccctgagcctgtacaacgaggaactggtgtctatgaacgtgcagggcgactacgagcccaccgacgccaccggcttcatcaacatcaacagcctgagactgaaagagtaccaccggctgcagtccaaagtgaccgccaag

SEQ ID NO: 3 - DNA, 1236 bp, Homo sapiens
  source 1..1236: mol_type = genomic DNA
atgtccagcaaaggctccgtggttctggcctacagtggcggcctggacacctcgtgcatcctcgtgtggctgaaggaacaaggctatgacgtcattgcctatctggccaacattggccagaaggaagacttcgaggaagccaggaagaaggcactgaagcttggggccaaaaaggtgttcattgaggatgtcagcagggagtttgtggaggagttcatctggccggccatccagtccagcgcactgtatgaggaccgctacctcctgggcacctctcttgccaggccctgcatcgcccgcaaacaagtggaaatcgcccagcgggagggggccaagtatgtgtcccacggcgccacaggaaaggggaacgatcaggtccggtttgagctcagctgctactcactggccccccagataaaggtcattgctccctggaggatgcctgaattctacaaccggttcaagggccgcaatgacctgatggagtacgcaaagcaacacgggattcccatcccggtcactcccaagaacccgtggagcatggatgagaacctcatgcacatcagctacgaggctggaatcctggagaaccccaagaaccaagcgcctccaggtctctacacgaagacccaggacccagccaaagcccccaacacccctgacattctcgagatcgagttcaaaaaaggggtccctgtgaaggtgaccaacgtcaaggatggcaccacccaccagacctccttggagctcttcatgtacctgaacgaagtcgcgggcaagcatggcgtgggccgtattgacatcgtggagaaccgcttcattggaatgaagtcccgaggtatctacgagaccccagcaggcaccatcctttaccacgctcatttagacatcgaggccttcaccatggaccgggaagtgcgcaaaatcaaacaaggcctgggcttgaaatttgctgagctggtgtataccggtttctggcacagccctgagtgtgaatttgtccgccactgcatcgccaagtcccaggagcgagtggaagggaaagtgcaggtgtccgtcctcaagggccaggtgtacatcctcggccgggagtccccactgtctctctacaatgaggagctggtgagcatgaacgtgcagggtgattatgagccaactgatgccaccgggttcatcaacatcaattccctcaggctgaaggaatatcatcgtctccagagcaaggtcactgccaaa

SEQ ID NO: 4 - DNA, 34 bp, synthetic construct
  misc_feature 1..34: note = constructed sequence
  source 1..34: mol_type = other DNA
tgtttgctgcttgcaatgtttgcccattttaggg

SEQ ID NO: 5 - DNA, 100 bp, synthetic construct
  misc_feature 1..100: note = constructed sequence
  source 1..100: mol_type = other DNA
ctacctcgtgatcgcccggcccctgttcaaacatgtcctaatactctgtctctgcaagggtcatcagtagttttccatcttactcaacatcctcccagtg

SEQ ID NO: 6 - DNA, 42 bp, synthetic construct
  misc_feature 1..42: note = constructed sequence
  source 1..42: mol_type = other DNA
aggttaatttttaaactgtttgctctggttaataatctcagg

SEQ ID NO: 7 - DNA, 322 bp, synthetic construct
  misc_feature 1..322: note = constructed sequence
  source 1..322: mol_type = other DNA
aaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggg

SEQ ID NO: 8 - DNA, 176 bp, synthetic construct
  misc_feature 1..176: note = constructed sequence
  source 1..176: mol_type = other DNA
actcaaagttcaaaccttatcattttttgctttgttcctcttggccttggttttgtacatcagctttgaaaataccatcccagggttaatgctggggttaatttataactaagagtgctctagttttgcaatacaggacatgctataaaaatggaaagatgttgctttctgagaga

SEQ ID NO: 9 - DNA, 477 bp, synthetic construct
  misc_feature 1..477: note = constructed sequence
  source 1..477: mol_type = other DNA
agggctggaagctacctttgacatcatttcctctgcgaatgcatgtataatttctacagaacctattagaaaggatcacccagcctctgcttttgtacaactttcccttaaaaaactgccaattccactgctgtttggcccaatagtgagaactttttcctgctgcctcttggtgcttttgcctatggcccctattctgcctgctgaagacactcttgccagcatggacttaaacccctccagctctgacaatcctctttctcttttgttttacatgaagggtctggcagccaaagcaatcactcaaagttcaaaccttatcattttttgctttgttcctcttggccttggttttgtacatcagctttgaaaataccatcccagggttaatgctggggttaatttataactaagagtgctctagttttgcaatacaggacatgctataaaaatggaaagatgttgctttctgagaga

SEQ ID NO: 10 - DNA, 218 bp, synthetic construct
  misc_feature 1..218: note = constructed sequence
  source 1..218: mol_type = other DNA
tggacacaggacgctgtggtttctgagccagggggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaata

SEQ ID NO: 11 - DNA, 190 bp, synthetic construct
  misc_feature 1..190: note = constructed sequence
  source 1..190: mol_type = other DNA
atttcatagaacgaatgttccgatgctctaatctctctagacaaggttcatatttgtatgggttacttattctctctttgttgactaagtcaataatcagaatcagcaggtttgcagtcagattggcagggataagcagcctagctcaggagaagtgagtataaaagccccaggctgggagcagccatca

SEQ ID NO: 12 - DNA, 215 bp, synthetic construct
  misc_feature 1..215: note = constructed sequence
  source 1..215: mol_type = other DNA
gcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctgggga

SEQ ID NO: 13 - DNA, 572 bp, synthetic construct
  misc_feature 1..572: note = constructed sequence
  source 1..572: mol_type = other DNA
agcttacttgtggtaccgagctcggatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattg

SEQ ID NO: 14 - DNA, 133 bp, synthetic construct
  misc_feature 1..133: note = constructed sequence
  source 1..133: mol_type = other DNA
gtaagtatcaaggttacaagacaggtttaaggagaccaatagaaactgggcttgtcgagacagagaagactcttgcgtttctgataggcacctattggtcttactgacatccactttgcctttctctccacag

SEQ ID NO: 15 - DNA, 542 bp, synthetic construct
  misc_feature 1..542: note = constructed sequence
  source 1..542: mol_type = other DNA
aatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcg

SEQ ID NO: 16 - DNA, 168 bp, synthetic construct
  misc_feature 1..168: note = constructed sequence
  source 1..168: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacg

SEQ ID NO: 17 - DNA, 168 bp, synthetic construct
  misc_feature 1..168: note = constructed sequence
  source 1..168: mol_type = other DNA
cgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 18 - DNA, 1421 bp, synthetic construct
  misc_feature 1..1421: note = constructed sequence
  source 1..1421: mol_type = other DNA
gtttgtttccttttttaaaatacattgagtatgcttgccttttagatatagaaatatctgatgctgtcttcttcactaaattttgattacatgatttgacagcaatattgaagagtctaacagccagcacgcaggttggtaagtactggttctttgttagctaggttttcttcttcttcatttttaaaactaaatagatcgacaatgcttatgatgcatttatgtttaataaacactgttcagttcatgatttggtcatgtaattcctgttagaaaacattcatctccttggtttaaaaaaattaaaagtgggaaaacaaagaaatagcagaatatagtgaaaaaaaataaccacattatttttgtttggacttaccactttgaaatcaaaatgggaaacaaaagcacaaacaatggccttatttacacaaaaagtctgattttaagatatatgacatttcaaggtttcagaagtatgtaatgaggtgtgtctctaattttttaaattatatatcttcaatttaaagttttagttaaaacataaagattaacctttcattagcaagctgttagttatcaccaaagcttttcatggattaggaaaaaatcattttgtctctatgtcaaacatcttggagttgatatttggggaaacacaatactcagttgagttccctaggggagaaaagcaagcttaagaattgacataaagagtaggaagttagctaatgcaacatatatcactttgttttttcacaactacagtgactttatgtatttcccagaggaaggcatacagggaagaaattatcccatttggacaaacagcatgttctcacaggaagcatttatcacacttacttgtcaactttctagaatcaaatctagtagctgacagtaccaggatcaggggtgccaaccctaagcacccccagaaagctgactggccctgtggttcccactccagacatgatgtcagctggaccataattaggcttctgttcttcaggagacatttgttcaaagtcatttgggcaaccatattctgaaaacagcccagccagggtgatggatcactttgcaaagatcctcaatgagctattttcaagtgatgacaaagtgtgaagttaaccgctcatttgagaactttctttttcatccaaagtaaattcaaatatgattagaaatctgaccttttattactggaattctcttgactaaaagtaaaattgaattttaattcctaaatctccatgtgtatacagtactgtgggaacatcacagattttggctccatgccctaaagagaaattggctttcagattatttggattaaaaacaaagactttcttaagagatgtaaaattttcatgatgttttcttttttgctaaaactaaagaattattcttttacatttcag

SEQ ID NO: 19 - protein, 738 aa, unidentified
  REGION 1..738: note = AAV8
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 20 - DNA, 735 bp, synthetic construct
  misc_feature 1..735: note = constructed sequence
  source 1..735: mol_type = other DNA
gctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggcgcgccgatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtagatctagcttacttgt

SEQ ID NO: 21 - DNA, 727 bp, synthetic construct
  misc_feature 1..727: note = constructed sequence
  source 1..727: mol_type = other DNA
agttaatttttaaaaagcagtcaaaagtccaagtgcccttgcgagcatttactctctctgtttgctctggttaataatctcaggagcacaaacattccttactagttctaggagttaatttttaaaaagcagtcaaaagtccaagtgcccttgcgagcatttactctctctgtttgctctggttaataatctcaggagcacaaacattccttactagttctagagcggccgccagtgtgctggaattcggcttttttagggctggaagctacctttgacatcatttcctctgcgaatgcatgtataatttctacagaacctattagaaaggatcacccagcctctgcttttgtacaactttcccttaaaaaactgccaatcccactgctgtttggcccaatagtgagaactttttcctgctgcctcttggtgcttttgcctatggcccctattctgcctgctgaagacactcttgccagcatggacttaaacccctccagctctgacaatcctctttctcttttgttttacatgaagggtctggcagccaaagcaatcactcaaagttcaaaccttatcattttttgctttgttcctcttggccttggttttgtacatcagctttgaaaataccatcccagggttaatgctggggttaatttataactgagagtgctctagttctgcaatacaggacatgctataaaaatggaaagatgttgctttc

SEQ ID NO: 22 - DNA, 6026 bp, synthetic construct
  misc_feature 1..6026: note = constructed sequence
  source 1..6026: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggcgcgccgatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtagatctagcttacttgtggtaccgagctcggatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgatctcgagtaactgaaggcggccgccaccatgagcagcaagggctctgtggtgctggcctactctggcggcctggacaccagctgtatcctcgtgtggctgaaagaacagggctacgacgtgatcgcctacctggccaacatcggccagaaagaggacttcgaggaagcccggaagaaggccctgaagctgggcgccaagaaggtgttcatcgaggacgtgtcccgcgagttcgtggaagagttcatctggcccgccatccagagcagcgccctgtacgaggacagatacctgctgggcaccagcctggccagaccctgtatcgcccggaaacaggtggaaatcgcccagcgcgagggcgccaaatacgtgtctcacggcgccaccggcaagggcaacgaccaggtgcgctttgagctgagctgctactccctggccccccagatcaaagtgatcgccccttggcggatgcccgagttctacaaccggttcaagggccggaacgacctgatggaatacgccaagcagcacggcatccccatccccgtgacccccaagaacccttggagcatggacgagaacctgatgcacatcagctacgaggccggcatcctggaaaaccccaagaatcaggcccctcccggcctgtacacaaagacccaggaccctgccaaggcccccaacacccccgacattctggaaatcgagttcaagaaaggcgtgcccgtgaaagtgaccaacgtgaaggacggcaccacccaccagacctccctggaactgttcatgtacctgaacgaggtggccggcaagcacggcgtgggcagaatcgacatcgtggaaaacagattcatcggcatgaagtcccggggcatctacgagacaccagccggcaccatcctgtaccacgcccacctggatatcgaggccttcaccatggaccgggaagtgcggaagatcaagcagggcctgggcctgaagttcgccgagctggtgtacacaggcttttggcacagccccgagtgcgagtttgtgcggcactgcattgccaagagccaggaacgggtggaaggcaaggtgcaggtgtccgtgctgaagggccaggtgtacattctgggcagagagagccccctgagcctgtacaacgaggaactggtgtctatgaacgtgcagggcgactacgagcccaccgacgccaccggcttcatcaacatcaacagcctgagactgaaagagtaccaccggctgcagtccaaagtgaccgccaagtgataagcatgcggatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagttaagggcgaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaagg

SEQ ID NO: 23 - DNA, 5141 bp, synthetic construct
  misc_feature 1..5141: note = constructed sequence
  source 1..5141: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagctagctgtttgctgcttgcaatgtttgcccattttagggtggacacaggacgctgtggtttctgagccagggggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccaggcggccgccaccatgagcagcaagggctctgtggtgctggcctactctggcggcctggacaccagctgtatcctcgtgtggctgaaagaacagggctacgacgtgatcgcctacctggccaacatcggccagaaagaggacttcgaggaagcccggaagaaggccctgaagctgggcgccaagaaggtgttcatcgaggacgtgtcccgcgagttcgtggaagagttcatctggcccgccatccagagcagcgccctgtacgaggacagatacctgctgggcaccagcctggccagaccctgtatcgcccggaaacaggtggaaatcgcccagcgcgagggcgccaaatacgtgtctcacggcgccaccggcaagggcaacgaccaggtgcgctttgagctgagctgctactccctggccccccagatcaaagtgatcgccccttggcggatgcccgagttctacaaccggttcaagggccggaacgacctgatggaatacgccaagcagcacggcatccccatccccgtgacccccaagaacccttggagcatggacgagaacctgatgcacatcagctacgaggccggcatcctggaaaaccccaagaatcaggcccctcccggcctgtacacaaagacccaggaccctgccaaggcccccaacacccccgacattctggaaatcgagttcaagaaaggcgtgcccgtgaaagtgaccaacgtgaaggacggcaccacccaccagacctccctggaactgttcatgtacctgaacgaggtggccggcaagcacggcgtgggcagaatcgacatcgtggaaaacagattcatcggcatgaagtcccggggcatctacgagacaccagccggcaccatcctgtaccacgcccacctggatatcgaggccttcaccatggaccgggaagtgcggaagatcaagcagggcctgggcctgaagttcgccgagctggtgtacacaggcttttggcacagccccgagtgcgagtttgtgcggcactgcattgccaagagccaggaacgggtggaaggcaaggtgcaggtgtccgtgctgaagggccaggtgtacattctgggcagagagagccccctgagcctgtacaacgaggaactggtgtctatgaacgtgcagggcgactacgagcccaccgacgccaccggcttcatcaacatcaacagcctgagactgaaagagtaccaccggctgcagtccaaagtgaccgccaagtgataagcatgcggatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagttaagggcgaattcccgattaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaagg

SEQ ID NO: 24 - DNA, 6071 bp, synthetic construct
  misc_feature 1..6071: note = constructed sequence
  source 1..6071: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagctaggggggatccactagtactcgagacctaggagttaatttttaaaaagcagtcaaaagtccaagtgcccttgcgagcatttactctctctgtttgctctggttaataatctcaggagcacaaacattccttactagttctaggagttaatttttaaaaagcagtcaaaagtccaagtgcccttgcgagcatttactctctctgtttgctctggttaataatctcaggagcacaaacattccttactagttctagagcggccgccagtgtgctggaattcggcttttttagggctggaagctacctttgacatcatttcctctgcgaatgcatgtataatttctacagaacctattagaaaggatcacccagcctctgcttttgtacaactttcccttaaaaaactgccaatcccactgctgtttggcccaatagtgagaactttttcctgctgcctcttggtgcttttgcctatggcccctattctgcctgctgaagacactcttgccagcatggacttaaacccctccagctctgacaatcctctttctcttttgttttacatgaagggtctggcagccaaagcaatcactcaaagttcaaaccttatcattttttgctttgttcctcttggccttggttttgtacatcagctttgaaaataccatcccagggttaatgctggggttaatttataactgagagtgctctagttctgcaatacaggacatgctataaaaatggaaagatgttgctttctgagagatcagcttacttgtggtaccgagctcggatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgatctcgagtaactgaaggcggccgccaccatgagcagcaagggctctgtggtgctggcctactctggcggcctggacaccagctgtatcctcgtgtggctgaaagaacagggctacgacgtgatcgcctacctggccaacatcggccagaaagaggacttcgaggaagcccggaagaaggccctgaagctgggcgccaagaaggtgttcatcgaggacgtgtcccgcgagttcgtggaagagttcatctggcccgccatccagagcagcgccctgtacgaggacagatacctgctgggcaccagcctggccagaccctgtatcgcccggaaacaggtggaaatcgcccagcgcgagggcgccaaatacgtgtctcacggcgccaccggcaagggcaacgaccaggtgcgctttgagctgagctgctactccctggccccccagatcaaagtgatcgccccttggcggatgcccgagttctacaaccggttcaagggccggaacgacctgatggaatacgccaagcagcacggcatccccatccccgtgacccccaagaacccttggagcatggacgagaacctgatgcacatcagctacgaggccggcatcctggaaaaccccaagaatcaggcccctcccggcctgtacacaaagacccaggaccctgccaaggcccccaacacccccgacattctggaaatcgagttcaagaaaggcgtgcccgtgaaagtgaccaacgtgaaggacggcaccacccaccagacctccctggaactgttcatgtacctgaacgaggtggccggcaagcacggcgtgggcagaatcgacatcgtggaaaacagattcatcggcatgaagtcccggggcatctacgagacaccagccggcaccatcctgtaccacgcccacctggatatcgaggccttcaccatggaccgggaagtgcggaagatcaagcagggcctgggcctgaagttcgccgagctggtgtacacaggcttttggcacagccccgagtgcgagtttgtgcggcactgcattgccaagagccaggaacgggtggaaggcaaggtgcaggtgtccgtgctgaagggccaggtgtacattctgggcagagagagccccctgagcctgtacaacgaggaactggtgtctatgaacgtgcagggcgactacgagcccaccgacgccaccggcttcatcaacatcaacagcctgagactgaaagagtaccaccggctgcagtccaaagtgaccgccaagtgataagcatgcggatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagttaagggcgaattcccgattaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaagg

SEQ ID NO: 25 - DNA, 6135 bp, synthetic construct
  misc_feature 1..6135: note = constructed sequence
  source 1..6135: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagctagcaggttaatttttaaaaagcagtcaaaagtccaagtggcccttggcagcatttactctctctgtttgctctggttaataatctcaggagcacaaacattccagatccaggttaatttttaaaaagcagtcaaaagtccaagtggcccttggcagcatttactctctctgtttgctctggttaataatctcaggagcacaaacattccagatccggcgcgccagggctggaagctacctttgacatcatttcctctgcgaatgcatgtataatttctacagaacctattagaaaggatcacccagcctctgcttttgtacaactttcccttaaaaaactgccaattccactgctgtttggcccaatagtgagaactttttcctgctgcctcttggtgcttttgcctatggcccctattctgcctgctgaagacactcttgccagcatggacttaaacccctccagctctgacaatcctctttctcttttgttttacatgaagggtctggcagccaaagcaatcactcaaagttcaaaccttatcattttttgctttgttcctcttggccttggttttgtacatcagctttgaaaataccatcccagggttaatgctggggttaatttataactaagagtgctctagttttgcaatacaggacatgctataaaaatggaaagatgttgctttctgagagactgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccaggcggccgccaccatgagcagcaagggctctgtggtgctggcctactctggcggcctggacaccagctgtatcctcgtgtggctgaaagaacagggctacgacgtgatcgcctacctggccaacatcggccagaaagaggacttcgaggaagcccggaagaaggccctgaagctgggcgccaagaaggtgttcatcgaggacgtgtcccgcgagttcgtggaagagttcatctggcccgccatccagagcagcgccctgtacgaggacagatacctgctgggcaccagcctggccagaccctgtatcgcccggaaacaggtggaaatcgcccagcgcgagggcgccaaatacgtgtctcacggcgccaccggcaagggcaacgaccaggtgcgctttgagctgagctgctactccctggccccccagatcaaagtgatcgccccttggcggatgcccgagttctacaaccggttcaagggccggaacgacctgatggaatacgccaagcagcacggcatccccatccccgtgacccccaagaacccttggagcatggacgagaacctgatgcacatcagctacgaggccggcatcctggaaaaccccaagaatcaggcccctcccggcctgtacacaaagacccaggaccctgccaaggcccccaacacccccgacattctggaaatcgagttcaagaaaggcgtgcccgtgaaagtgaccaacgtgaaggacggcaccacccaccagacctccctggaactgttcatgtacctgaacgaggtggccggcaagcacggcgtgggcagaatcgacatcgtggaaaacagattcatcggcatgaagtcccggggcatctacgagacaccagccggcaccatcctgtaccacgcccacctggatatcgaggccttcaccatggaccgggaagtgcggaagatcaagcagggcctgggcctgaagttcgccgagctggtgtacacaggcttttggcacagccccgagtgcgagtttgtgcggcactgcattgccaagagccaggaacgggtggaaggcaaggtgcaggtgtccgtgctgaagggccaggtgtacattctgggcagagagagccccctgagcctgtacaacgaggaactggtgtctatgaacgtgcagggcgactacgagcccaccgacgccaccggcttcatcaacatcaacagcctgagactgaaagagtaccaccggctgcagtccaaagtgaccgccaagtgataagcatgcggatccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagttaagggcgaattcccgattaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaagg

SEQ ID NO: 26 - DNA, 5587 bp, synthetic construct
  misc_feature 1..5587: note = constructed sequence
  source 1..5587: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagctagcaggttaatttttaaaaagcagtcaaaagtccaagtggcccttggcagcatttactctctctgtttgctctggttaataatctcaggagcacaaacattccagatccaggttaatttttaaaaagcagtcaaaagtccaagtggcccttggcagcatttactctctctgtttgctctggttaataatctcaggagcacaaacattccagatccggcgcgccagggctggaagctacctttgacatcatttcctctgcgaatgcatgtataatttctacagaacctattagaaaggatcacccagcctctgcttttgtacaactttcccttaaaaaactgccaattccactgctgtttggcccaatagtgagaactttttcctgctgcctcttggtgcttttgcctatggcccctattctgcctgctgaagacactcttgccagcatggacttaaacccctccagctctgacaatcctctttctcttttgttttacatgaagggtctggcagccaaagcaatcactcaaagttcaaaccttatcattttttgctttgttcctcttggccttggttttgtacatcagctttgaaaataccatcccagggttaatgctggggttaatttataactaagagtgctctagttttgcaatacaggacatgctataaaaatggaaagatgttgctttctgagagactgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccaggcggccgccaccatgagcagcaagggctctgtggtgctggcctactctggcggcctggacaccagctgtatcctcgtgtggctgaaagaacagggctacgacgtgatcgcctacctggccaacatcggccagaaagaggacttcgaggaagcccggaagaaggccctgaagctgggcgccaagaaggtgttcatcgaggacgtgtcccgcgagttcgtggaagagttcatctggcccgccatccagagcagcgccctgtacgaggacagatacctgctgggcaccagcctggccagaccctgtatcgcccggaaacaggtggaaatcgcccagcgcgagggcgccaaatacgtgtctcacggcgccaccggcaagggcaacgaccaggtgcgctttgagctgagctgctactccctggccccccagatcaaagtgatcgccccttggcggatgcccgagttctacaaccggttcaagggccggaacgacctgatggaatacgccaagcagcacggcatccccatccccgtgacccccaagaacccttggagcatggacgagaacctgatgcacatcagctacgaggccggcatcctggaaaaccccaagaatcaggcccctcccggcctgtacacaaagacccaggaccctgccaaggcccccaacacccccgacattctggaaatcgagttcaagaaaggcgtgcccgtgaaagtgaccaacgtgaaggacggcaccacccaccagacctccctggaactgttcatgtacctgaacgaggtggccggcaagcacggcgtgggcagaatcgacatcgtggaaaacagattcatcggcatgaagtcccggggcatctacgagacaccagccggcaccatcctgtaccacgcccacctggatatcgaggccttcaccatggaccgggaagtgcggaagatcaagcagggcctgggcctgaagttcgccgagctggtgtacacaggcttttggcacagccccgagtgcgagtttgtgcggcactgcattgccaagagccaggaacgggtggaaggcaaggtgcaggtgtccgtgctgaagggccaggtgtacattctgggcagagagagccccctgagcctgtacaacgaggaactggtgtctatgaacgtgcagggcgactacgagcccaccgacgccaccggcttcatcaacatcaacagcctgagactgaaagagtaccaccggctgcagtccaaagtgaccgccaagtgataagcatgcggatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagttaagggcgaattcccgattaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaagg

SEQ ID NO: 27 - DNA, 5587 bp, synthetic construct
  misc_feature 1..5587: note = constructed sequence
  source 1..5587: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagctagcaggttaatttttaaaaagcagtcaaaagtccaagtggcccttggcagcatttactctctctgtttgctctggttaataatctcaggagcacaaacattccagatccaggttaatttttaaaaagcagtcaaaagtccaagtggcccttggcagcatttactctctctgtttgctctggttaataatctcaggagcacaaacattccagatccggcgcgccagggctggaagctacctttgacatcatttcctctgcgaatgcatgtataatttctacagaacctattagaaaggatcacccagcctctgcttttgtacaactttcccttaaaaaactgccaattccactgctgtttggcccaatagtgagaactttttcctgctgcctcttggtgcttttgcctatggcccctattctgcctgctgaagacactcttgccagcatggacttaaacccctccagctctgacaatcctctttctcttttgttttacatgaagggtctggcagccaaagcaatcactcaaagttcaaaccttatcattttttgctttgttcctcttggccttggttttgtacatcagctttgaaaataccatcccagggttaatgctggggttaatttataactaagagtgctctagttttgcaatacaggacatgctataaaaatggaaagatgttgctttctgagagactgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccaggcggccgccaccatgtccagcaaaggctccgtggttctggcctacagtggcggcctggacacctcgtgcatcctcgtgtggctgaaggaacaaggctatgacgtcattgcctatctggccaacattggccagaaggaagacttcgaggaagccaggaagaaggcactgaagcttggggccaaaaaggtgttcattgaggatgtcagcagggagtttgtggaggagttcatctggccggccatccagtccagcgcactgtatgaggaccgctacctcctgggcacctctcttgccaggccctgcatcgcccgcaaacaagtggaaatcgcccagcgggagggggccaagtatgtgtcccacggcgccacaggaaaggggaacgatcaggtccggtttgagctcagctgctactcactggccccccagataaaggtcattgctccctggaggatgcctgaattctacaaccggttcaagggccgcaatgacctgatggagtacgcaaagcaacacgggattcccatcccggtcactcccaagaacccgtggagcatggatgagaacctcatgcacatcagctacgaggctggaatcctggagaaccccaagaaccaagcgcctccaggtctctacacgaagacccaggacccagccaaagcccccaacacccctgacattctcgagatcgagttcaaaaaaggggtccctgtgaaggtgaccaacgtcaaggatggcaccacccaccagacctccttggagctcttcatgtacctgaacgaagtcgcgggcaagcatggcgtgggccgtattgacatcgtggagaaccgcttcattggaatgaagtcccgaggtatctacgagaccccagcaggcaccatcctttaccacgctcatttagacatcgaggccttcaccatggaccgggaagtgcgcaaaatcaaacaaggcctgggcttgaaatttgctgagctggtgtataccggtttctggcacagccctgagtgtgaatttgtccgccactgcatcgccaagtcccaggagcgagtggaagggaaagtgcaggtgtccgtcctcaagggccaggtgtacatcctcggccgggagtccccactgtctctctacaatgaggagctggtgagcatgaacgtgcagggtgattatgagccaactgatgccaccgggttcatcaacatcaattccctcaggctgaaggaatatcatcgtctccagagcaaggtcactgccaaatgataagcatgcggatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagttaagggcgaattcccgattaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaagg

SEQ ID NO: 28 - DNA, 6026 bp, synthetic construct
  misc_feature 1..6026: note = constructed sequence
  source 1..6026: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggcgcgccgatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtagatctagcttacttgtggtaccgagctcggatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgatctcgagtaactgaaggcggccgccaccatgtccagcaaaggctccgtggttctggcctacagtggcggcctggacacctcgtgcatcctcgtgtggctgaaggaacaaggctatgacgtcattgcctatctggccaacattggccagaaggaagacttcgaggaagccaggaagaaggcactgaagcttggggccaaaaaggtgttcattgaggatgtcagcagggagtttgtggaggagttcatctggccggccatccagtccagcgcactgtatgaggaccgctacctcctgggcacctctcttgccaggccctgcatcgcccgcaaacaagtggaaatcgcccagcgggagggggccaagtatgtgtcccacggcgccacaggaaaggggaacgatcaggtccggtttgagctcagctgctactcactggccccccagataaaggtcattgctccctggaggatgcctgaattctacaaccggttcaagggccgcaatgacctgatggagtacgcaaagcaacacgggattcccatcccggtcactcccaagaacccgtggagcatggatgagaacctcatgcacatcagctacgaggctggaatcctggagaaccccaagaaccaagcgcctccaggtctctacacgaagacccaggacccagccaaagcccccaacacccctgacattctcgagatcgagttcaaaaaaggggtccctgtgaaggtgaccaacgtcaaggatggcaccacccaccagacctccttggagctcttcatgtacctgaacgaagtcgcgggcaagcatggcgtgggccgtattgacatcgtggagaaccgcttcattggaatgaagtcccgaggtatctacgagaccccagcaggcaccatcctttaccacgctcatttagacatcgaggccttcaccatggaccgggaagtgcgcaaaatcaaacaaggcctgggcttgaaatttgctgagctggtgtataccggtttctggcacagccctgagtgtgaatttgtccgccactgcatcgccaagtcccaggagcgagtggaagggaaagtgcaggtgtccgtcctcaagggccaggtgtacatcctcggccgggagtccccactgtctctctacaatgaggagctggtgagcatgaacgtgcagggtgattatgagccaactgatgccaccgggttcatcaacatcaattccctcaggctgaaggaatatcatcgtctccagagcaaggtcactgccaaatgataagcatgcggatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagttaagggcgaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaagg

SEQ ID NO: 29 - DNA, 5876 bp, synthetic construct
  misc_feature 1..5876: note = constructed sequence
  source 1..5876: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagctagccgctgacaaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggagatccggcgcgcctggacacaggacgctgtggtttctgagccagggggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatagcgagatctagcttacttgtggtaccagctcggatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgatctcgagtaactgaaggcggccgccaccatgagcagcaagggctctgtggtgctggcctactctggcggcctggacaccagctgtatcctcgtgtggctgaaagaacagggctacgacgtgatcgcctacctggccaacatcggccagaaagaggacttcgaggaagcccggaagaaggccctgaagctgggcgccaagaaggtgttcatcgaggacgtgtcccgcgagttcgtggaagagttcatctggcccgccatccagagcagcgccctgtacgaggacagatacctgctgggcaccagcctggccagaccctgtatcgcccggaaacaggtggaaatcgcccagcgcgagggcgccaaatacgtgtctcacggcgccaccggcaagggcaacgaccaggtgcgctttgagctgagctgctactccctggccccccagatcaaagtgatcgccccttggcggatgcccgagttctacaaccggttcaagggccggaacgacctgatggaatacgccaagcagcacggcatccccatccccgtgacccccaagaacccttggagcatggacgagaacctgatgcacatcagctacgaggccggcatcctggaaaaccccaagaatcaggcccctcccggcctgtacacaaagacccaggaccctgccaaggcccccaacacccccgacattctggaaatcgagttcaagaaaggcgtgcccgtgaaagtgaccaacgtgaaggacggcaccacccaccagacctccctggaactgttcatgtacctgaacgaggtggccggcaagcacggcgtgggcagaatcgacatcgtggaaaacagattcatcggcatgaagtcccggggcatctacgagacaccagccggcaccatcctgtaccacgcccacctggatatcgaggccttcaccatggaccgggaagtgcggaagatcaagcagggcctgggcctgaagttcgccgagctggtgtacacaggcttttggcacagccccgagtgcgagtttgtgcggcactgcattgccaagagccaggaacgggtggaaggcaaggtgcaggtgtccgtgctgaagggccaggtgtacattctgggcagagagagccccctgagcctgtacaacgaggaactggtgtctatgaacgtgcagggcgactacgagcccaccgacgccaccggcttcatcaacatcaacagcctgagactgaaagagtaccaccggctgcagtccaaagtgaccgccaagtgataagcatgcggatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagttaagggcgaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaagg

SEQ ID NO: 30 - DNA, 4893 bp, synthetic construct
  misc_feature 1..4893: note = constructed sequence
  source 1..4893: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacttaagtgtttgctgcttgcaatgtttgcccattttaggggaattctggacacaggacgctgtggtttctgagccagggggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatagcggccgccaccatgagcagcaagggctctgtggtgctggcctactctggcggcctggacaccagctgtatcctcgtgtggctgaaagaacagggctacgacgtgatcgcctacctggccaacatcggccagaaagaggacttcgaggaagcccggaagaaggccctgaagctgggcgccaagaaggtgttcatcgaggacgtgtcccgcgagttcgtggaagagttcatctggcccgccatccagagcagcgccctgtacgaggacagatacctgctgggcaccagcctggccagaccctgtatcgcccggaaacaggtggaaatcgcccagcgcgagggcgccaaatacgtgtctcacggcgccaccggcaagggcaacgaccaggtgcgctttgagctgagctgctactccctggccccccagatcaaagtgatcgccccttggcggatgcccgagttctacaaccggttcaagggccggaacgacctgatggaatacgccaagcagcacggcatccccatccccgtgacccccaagaacccttggagcatggacgagaacctgatgcacatcagctacgaggccggcatcctggaaaaccccaagaatcaggcccctcccggcctgtacacaaagacccaggaccctgccaaggcccccaacacccccgacattctggaaatcgagttcaagaaaggcgtgcccgtgaaagtgaccaacgtgaaggacggcaccacccaccagacctccctggaactgttcatgtacctgaacgaggtggccggcaagcacggcgtgggcagaatcgacatcgtggaaaacagattcatcggcatgaagtcccggggcatctacgagacaccagccggcaccatcctgtaccacgcccacctggatatcgaggccttcaccatggaccgggaagtgcggaagatcaagcagggcctgggcctgaagttcgccgagctggtgtacacaggcttttggcacagccccgagtgcgagtttgtgcggcactgcattgccaagagccaggaacgggtggaaggcaaggtgcaggtgtccgtgctgaagggccaggtgtacattctgggcagagagagccccctgagcctgtacaacgaggaactggtgtctatgaacgtgcagggcgactacgagcccaccgacgccaccggcttcatcaacatcaacagcctgagactgaaagagtaccaccggctgcagtccaaagtgaccgccaagtgataagcatgcggatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaagg

SEQ ID NO: 31 - DNA, 4931 bp, synthetic construct
  misc_feature 1..4931: note = constructed sequence
  source 1..4931: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacttaagctacctcgtgatcgcccggcccctgttcaaacatgtcctaatactctgtctctgcaagggtcatcagtagttttccatcttactcaacatcctcccagtggaattcatttcatagaacgaatgttccgatgctctaatctctctagacaaggttcatatttgtatgggttacttattctctctttgttgactaagtcaataatcagaatcagcaggtttgcagtcagattggcagggataagcagcctagctcaggagaagtgagtataaaagccccaggctgggagcagccatcagcggccgccaccatgagcagcaagggctctgtggtgctggcctactctggcggcctggacaccagctgtatcctcgtgtggctgaaagaacagggctacgacgtgatcgcctacctggccaacatcggccagaaagaggacttcgaggaagcccggaagaaggccctgaagctgggcgccaagaaggtgttcatcgaggacgtgtcccgcgagttcgtggaagagttcatctggcccgccatccagagcagcgccctgtacgaggacagatacctgctgggcaccagcctggccagaccctgtatcgcccggaaacaggtggaaatcgcccagcgcgagggcgccaaatacgtgtctcacggcgccaccggcaagggcaacgaccaggtgcgctttgagctgagctgctactccctggccccccagatcaaagtgatcgccccttggcggatgcccgagttctacaaccggttcaagggccggaacgacctgatggaatacgccaagcagcacggcatccccatccccgtgacccccaagaacccttggagcatggacgagaacctgatgcacatcagctacgaggccggcatcctggaaaaccccaagaatcaggcccctcccggcctgtacacaaagacccaggaccctgccaaggcccccaacacccccgacattctggaaatcgagttcaagaaaggcgtgcccgtgaaagtgaccaacgtgaaggacggcaccacccaccagacctccctggaactgttcatgtacctgaacgaggtggccggcaagcacggcgtgggcagaatcgacatcgtggaaaacagattcatcggcatgaagtcccggggcatctacgagacaccagccggcaccatcctgtaccacgcccacctggatatcgaggccttcaccatggaccgggaagtgcggaagatcaagcagggcctgggcctgaagttcgccgagctggtgtacacaggcttttggcacagccccgagtgcgagtttgtgcggcactgcattgccaagagccaggaacgggtggaaggcaaggtgcaggtgtccgtgctgaagggccaggtgtacattctgggcagagagagccccctgagcctgtacaacgaggaactggtgtctatgaacgtgcagggcgactacgagcccaccgacgccaccggcttcatcaacatcaacagcctgagactgaaagagtaccaccggctgcagtccaaagtgaccgccaagtgataagcatgcggatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaagg

SEQ ID NO: 32 - DNA, 6031 bp, synthetic construct
  misc_feature 1..6031: note = constructed sequence
  source 1..6031: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagctaggagttaatttttaaaaagcagtcaaaagtccaagtgcccttgcgagcatttactctctctgtttgctctggttaataatctcaggagcacaaacattccttactagttctaggagttaatttttaaaaagcagtcaaaagtccaagtgcccttgcgagcatttactctctctgtttgctctggttaataatctcaggagcacaaacattccttactagttctagagcggccgccagtgtgctggaattcggcttttttagggctggaagctacctttgacatcatttcctctgcgaatgcatgtataatttctacagaacctattagaaaggatcacccagcctctgcttttgtacaactttcccttaaaaaactgccaatcccactgctgtttggcccaatagtgagaactttttcctgctgcctcttggtgcttttgcctatggcccctattctgcctgctgaagacactcttgccagcatggacttaaacccctccagctctgacaatcctctttctcttttgttttacatgaagggtctggcagccaaagcaatcactcaaagttcaaaccttatcattttttgctttgttcctcttggccttggttttgtacatcagctttgaaaataccatcccagggttaatgctggggttaatttataactgagagtgctctagttctgcaatacaggacatgctataaaaatggaaagatgttgctttctgagagatcagcttacttgtggtaccgagctcggatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgatctcgaggccgccaccatgtccagcaaaggctccgtggttctggcctacagtggcggcctggacacctcgtgcatcctcgtgtggctgaaggaacaaggctatgacgtcattgcctatctggccaacattggccagaaggaagacttcgaggaagccaggaagaaggcactgaagcttggggccaaaaaggtgttcattgaggatgtcagcagggagtttgtggaggagttcatctggccggccatccagtccagcgcactgtatgaggaccgctacctcctgggcacctctcttgccaggccctgcatcgcccgcaaacaagtggaaatcgcccagcgggagggggccaagtatgtgtcccacggcgccacaggaaaggggaacgatcaggtccggtttgagctcagctgctactcactggccccccagataaaggtcattgctccctggaggatgcctgaattctacaaccggttcaagggccgcaatgacctgatggagtacgcaaagcaacacgggattcccatcccggtcactcccaagaacccgtggagcatggatgagaacctcatgcacatcagctacgaggctggaatcctggagaaccccaagaaccaagcgcctccaggtctctacacgaagacccaggacccagccaaagcccccaacacccctgacattctcgagatcgagttcaaaaaaggggtccctgtgaaggtgaccaacgtcaaggatggcaccacccaccagacctccttggagctcttcatgtacctgaacgaagtcgcgggcaagcatggcgtgggccgtattgacatcgtggagaaccgcttcattggaatgaagtcccgaggtatctacgagaccccagcaggcaccatcctttaccacgctcatttagacatcgaggccttcaccatggaccgggaagtgcgcaaaatcaaacaaggcctgggcttgaaatttgctgagctggtgtataccggtttctggcacagccctgagtgtgaatttgtccgccactgcatcgccaagtcccaggagcgagtggaagggaaagtgcaggtgtccgtcctcaagggccaggtgtacatcctcggccgggagtccccactgtctctctacaatgaggagctggtgagcatgaacgtgcagggtgattatgagccaactgatgccaccgggttcatcaacatcaattccctcaggctgaaggaatatcatcgtctccagagcaaggtcactgccaaatgataagcatgcggatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagttaagggcgaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaagg

SEQ ID NO: 33 - DNA, 6850 bp, synthetic construct
  misc_feature 1..6850: note = constructed sequence
  source 1..6850: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagctagcggatccaggttaatttttaaaaagcagtcaaaagtccaagtggcccttggcagcatttactctctctgtttgctctggttaataatctcaggagcacaaacattccagatccaggttaatttttaaaaagcagtcaaaagtccaagtggcccttggcagcatttactctctctgtttgctctggttaataatctcaggagcacaaacattccagatccggcgcgccagggctggaagctacctttgacatcatttcctctgcgaatgcatgtataatttctacagaacctattagaaaggatcacccagcctctgcttttgtacaactttcccttaaaaaactgccaattccactgctgtttggcccaatagtgagaactttttcctgctgcctcttggtgcttttgcctatggcccctattctgcctgctgaagacactcttgccagcatggacttaaacccctccagctctgacaatcctctttctcttttgttttacatgaagggtctggcagccaaagcaatcactcaaagttcaaaccttatcattttttgctttgttcctcttggccttggttttgtacatcagctttgaaaataccatcccagggttaatgctggggttaatttataactaagagtgctctagttttgcaatacaggacatgctataaaaatggaaagatgttgctttctgagagagcggccgcgtttgtttccttttttaaaatacattgagtatgcttgccttttagatatagaaatatctgatgctgtcttcttcactaaattttgattacatgatttgacagcaatattgaagagtctaacagccagcacgcaggttggtaagtactggttctttgttagctaggttttcttcttcttcatttttaaaactaaatagatcgacaatgcttatgatgcatttatgtttaataaacactgttcagttcatgatttggtcatgtaattcctgttagaaaacattcatctccttggtttaaaaaaattaaaagtgggaaaacaaagaaatagcagaatatagtgaaaaaaaataaccacattatttttgtttggacttaccactttgaaatcaaaatgggaaacaaaagcacaaacaatggccttatttacacaaaaagtctgattttaagatatatgacatttcaaggtttcagaagtatgtaatgaggtgtgtctctaattttttaaattatatatcttcaatttaaagttttagttaaaacataaagattaacctttcattagcaagctgttagttatcaccaaagcttttcatggattaggaaaaaatcattttgtctctatgtcaaacatcttggagttgatatttggggaaacacaatactcagttgagttccctaggggagaaaagcaagcttaagaattgacataaagagtaggaagttagctaatgcaacatatatcactttgttttttcacaactacagtgactttatgtatttcccagaggaaggcatacagggaagaaattatcccatttggacaaacagcatgttctcacaggaagcatttatcacacttacttgtcaactttctagaatcaaatctagtagctgacagtaccaggatcaggggtgccaaccctaagcacccccagaaagctgactggccctgtggttcccactccagacatgatgtcagctggaccataattaggcttctgttcttcaggagacatttgttcaaagtcatttgggcaaccatattctgaaaacagcccagccagggtgatggatcactttgcaaagatcctcaatgagctattttcaagtgatgacaaagtgtgaagttaaccgctcatttgagaactttctttttcatccaaagtaaattcaaatatgattagaaatctgaccttttattactggaattctcttgactaaaagtaaaattgaattttaattcctaaatctccatgtgtatacagtactgtgggaacatcacagattttggctccatgccctaaagagaaattggctttcagattatttggattaaaaacaaagactttcttaagagatgtaaaattttcatgatgttttcttttttgctaaaactaaagaattattcttttacatttcagatggccgccaccatgagcagcaagggctctgtggtgctggcctactctggcggcctggacaccagctgtatcctcgtgtggctgaaagaacagggctacgacgtgatcgcctacctggccaacatcggccagaaagaggacttcgaggaagcccggaagaaggccctgaagctgggcgccaagaaggtgttcatcgaggacgtgtcccgcgagttcgtggaagagttcatctggcccgccatccagagcagcgccctgtacgaggacagatacctgctgggcaccagcctggccagaccctgtatcgcccggaaacaggtggaaatcgcccagcgcgagggcgccaaatacgtgtctcacggcgccaccggcaagggcaacgaccaggtgcgctttgagctgagctgctactccctggccccccagatcaaagtgatcgccccttggcggatgcccgagttctacaaccggttcaagggccggaacgacctgatggaatacgccaagcagcacggcatccccatccccgtgacccccaagaacccttggagcatggacgagaacctgatgcacatcagctacgaggccggcatcctggaaaaccccaagaatcaggcccctcccggcctgtacacaaagacccaggaccctgccaaggcccccaacacccccgacattctggaaatcgagttcaagaaaggcgtgcccgtgaaagtgaccaacgtgaaggacggcaccacccaccagacctccctggaactgttcatgtacctgaacgaggtggccggcaagcacggcgtgggcagaatcgacatcgtggaaaacagattcatcggcatgaagtcccggggcatctacgagacaccagccggcaccatcctgtaccacgcccacctggatatcgaggccttcaccatggaccgggaagtgcggaagatcaagcagggcctgggcctgaagttcgccgagctggtgtacacaggcttttggcacagccccgagtgcgagtttgtgcggcactgcattgccaagagccaggaacgggtggaaggcaaggtgcaggtgtccgtgctgaagggccaggtgtacattctgggcagagagagccccctgagcctgtacaacgaggaactggtgtctatgaacgtgcagggcgactacgagcccaccgacgccaccggcttcatcaacatcaacagcctgagactgaaagagtaccaccggctgcagtccaaagtgaccgccaagtgataagcatgcggatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagttaagggcgaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaagg

SEQ ID NO: 34 - DNA, 5608 bp, synthetic construct
  misc_feature 1..5608: note = constructed sequence
  source 1..5608: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagctacctcgtgatcgcccggcccctgttcaaacatgtcctaatactctgtctctgcaagggtcatcagtagttttccatcttactcaacatcctcccagtggaattcatttcatagaacgaatgttccgatgctctaatctctctagacaaggttcatatttgtatgggttacttattctctctttgttgactaagtcaataatcagaatcagcaggtttgcagtcagattggcagggataagcagcctagctcaggagaagtgagtataaaagccccaggctgggagcagccatcagcggccgatctagcttacttgtggtaccagctcggatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgatctcgagtaactgaaggcggccgccaccatgtccagcaaaggctccgtggttctggcctacagtggcggcctggacacctcgtgcatcctcgtgtggctgaaggaacaaggctatgacgtcattgcctatctggccaacattggccagaaggaagacttcgaggaagccaggaagaaggcactgaagcttggggccaaaaaggtgttcattgaggatgtcagcagggagtttgtggaggagttcatctggccggccatccagtccagcgcactgtatgaggaccgctacctcctgggcacctctcttgccaggccctgcatcgcccgcaaacaagtggaaatcgcccagcgggagggggccaagtatgtgtcccacggcgccacaggaaaggggaacgatcaggtccggtttgagctcagctgctactcactggccccccagataaaggtcattgctccctggaggatgcctgaattctacaaccggttcaagggccgcaatgacctgatggagtacgcaaagcaacacgggattcccatcccggtcactcccaagaacccgtggagcatggatgagaacctcatgcacatcagctacgaggctggaatcctggagaaccccaagaaccaagcgcctccaggtctctacacgaagacccaggacccagccaaagcccccaacacccctgacattctcgagatcgagttcaaaaaaggggtccctgtgaaggtgaccaacgtcaaggatggcaccacccaccagacctccttggagctcttcatgtacctgaacgaagtcgcgggcaagcatggcgtgggccgtattgacatcgtggagaaccgcttcattggaatgaagtcccgaggtatctacgagaccccagcaggcaccatcctttaccacgctcatttagacatcgaggccttcaccatggaccgggaagtgcgcaaaatcaaacaaggcctgggcttgaaatttgctgagctggtgtataccggtttctggcacagccctgagtgtgaatttgtccgccactgcatcgccaagtcccaggagcgagtggaagggaaagtgcaggtgtccgtcctcaagggccaggtgtacatcctcggccgggagtccccactgtctctctacaatgaggagctggtgagcatgaacgtgcagggtgattatgagccaactgatgccaccgggttcatcaacatcaattccctcaggctgaaggaatatcatcgtctccagagcaaggtcactgccaaatgataagcatgcggatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagttaagggcgaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaagg

SEQ ID NO: 35 - DNA, 5876 bp, synthetic construct
  misc_feature 1..5876: note = constructed sequence
  source 1..5876: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagctagccgctgacaaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggagatccggcgcgcctggacacaggacgctgtggtttctgagccagggggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatagcgagatctagcttacttgtggtaccagctcggatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgatctcgagtaactgaaggcggccgccaccatgtccagcaaaggctccgtggttctggcctacagtggcggcctggacacctcgtgcatcctcgtgtggctgaaggaacaaggctatgacgtcattgcctatctggccaacattggccagaaggaagacttcgaggaagccaggaagaaggcactgaagcttggggccaaaaaggtgttcattgaggatgtcagcagggagtttgtggaggagttcatctggccggccatccagtccagcgcactgtatgaggaccgctacctcctgggcacctctcttgccaggccctgcatcgcccgcaaacaagtggaaatcgcccagcgggagggggccaagtatgtgtcccacggcgccacaggaaaggggaacgatcaggtccggtttgagctcagctgctactcactggccccccagataaaggtcattgctccctggaggatgcctgaattctacaaccggttcaagggccgcaatgacctgatggagtacgcaaagcaacacgggattcccatcccggtcactcccaagaacccgtggagcatggatgagaacctcatgcacatcagctacgaggctggaatcctggagaaccccaagaaccaagcgcctccaggtctctacacgaagacccaggacccagccaaagcccccaacacccctgacattctcgagatcgagttcaaaaaaggggtccctgtgaaggtgaccaacgtcaaggatggcaccacccaccagacctccttggagctcttcatgtacctgaacgaagtcgcgggcaagcatggcgtgggccgtattgacatcgtggagaaccgcttcattggaatgaagtcccgaggtatctacgagaccccagcaggcaccatcctttaccacgctcatttagacatcgaggccttcaccatggaccgggaagtgcgcaaaatcaaacaaggcctgggcttgaaatttgctgagctggtgtataccggtttctggcacagccctgagtgtgaatttgtccgccactgcatcgccaagtcccaggagcgagtggaagggaaagtgcaggtgtccgtcctcaagggccaggtgtacatcctcggccgggagtccccactgtctctctacaatgaggagctggtgagcatgaacgtgcagggtgattatgagccaactgatgccaccgggttcatcaacatcaattccctcaggctgaaggaatatcatcgtctccagagcaaggtcactgccaaatgataagcatgcggatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagttaagggcgaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaagg

SEQ ID NO: 36 - DNA, 4393 bp, adeno-associated virus 8
  source 1..4393: mol_type = genomic DNA
cagagagggagtggccaactccatcactaggggtagcgcgaagcgcctcccacgctgccgcgtcagcgctgacgtaaattacgtcataggggagtggtcctgtattagctgtcacgtgagtgcttttgcggcattttgcgacaccacgtggccatttgaggtatatatggccgagtgagcgagcaggatctccattttgaccgcgaaatttgaacgagcagcagccatgccgggcttctacgagatcgtgatcaaggtgccgagcgacctggacgagcacctgccgggcatttctgactcgtttgtgaactgggtggccgagaaggaatgggagctgcccccggattctgacatggatcggaatctgatcgagcaggcacccctgaccgtggccgagaagctgcagcgcgacttcctggtccaatggcgccgcgtgagtaaggccccggaggccctcttctttgttcagttcgagaagggcgagagctactttcacctgcacgttctggtcgagaccacgggggtcaagtccatggtgctaggccgcttcctgagtcagattcgggaaaagcttggtccagaccatctacccgcggggtcgagccccaccttgcccaactggttcgcggtgaccaaagacgcggtaatggcgccggcgggggggaacaaggtggtggacgagtgctacatccccaactacctcctgcccaagactcagcccgagctgcagtgggcgtggactaacatggaggagtatataagcgcgtgcttgaacctggccgagcgcaaacggctcgtggcgcagcacctgacccacgtcagccagacgcaggagcagaacaaggagaatctgaaccccaattctgacgcgcccgtgatcaggtcaaaaacctccgcgcgctatatggagctggtcgggtggctggtggaccggggcatcacctccgagaagcagtggatccaggaggaccaggcctcgtacatctccttcaacgccgcctccaactcgcggtcccagatcaaggccgcgctggacaatgccggcaagatcatggcgctgaccaaatccgcgcccgactacctggtggggccctcgctgcccgcggacattacccagaaccgcatctaccgcatcctcgctctcaacggctacgaccctgcctacgccggctccgtctttctcggctgggctcagaaaaagttcgggaaacgcaacaccatctggctgtttggacccgccaccaccggcaagaccaacattgcggaagccatcgcccacgccgtgcccttctacggctgcgtcaactggaccaatgagaactttcccttcaatgattgcgtcgacaagatggtgatctggtgggaggagggcaagatgacggccaaggtcgtggagtccgccaaggccattctcggcggcagcaaggtgcgcgtggaccaaaagtgcaagtcgtccgcccagatcgaccccacccccgtgatcgtcacctccaacaccaacatgtgcgccgtgattgacgggaacagcaccaccttcgagcaccagcagcctctccaggaccggatgtttaagttcgaactcacccgccgtctggagcacgactttggcaaggtgacaaagcaggaagtcaaagagttcttccgctgggccagtgatcacgtgaccgaggtggcgcatgagttttacgtcagaaagggcggagccagcaaaagacccgcccccgatgacgcggataaaagcgagcccaagcgggcctgcccctcagtcgcggatccatcgacgtcagacgcggaaggagctccggtggactttgccgacaggtaccaaaacaaatgttctcgtcacgcgggcatgcttcagatgctgtttccctgcaaaacgtgcgagagaatgaatcagaatttcaacatttgcttcacacacggggtcagagactgctcagagtgtttccccggcgtgtcagaatctcaaccggtcgtcagaaagaggacgtatcggaaactctgtgcgattcatcatctgctggggcgggctcccgagattgcttgctcggcctgcgatctggtcaacgtggacctggatgactgtgtttctgagcaataaatgacttaaaccaggtatggctgccgatggttatcttccagattggctcgaggacaacctctctgagggcattcgcgagtggtgggcgctgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctgcaggcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccatcaccccagcgttctccagactcctctacgggcatcggcaagaaaggccaacagcccgccagaaaaagactcaattttggtcagactggcgactcagagtcagttccagaccctcaacctctcggagaacctccagcagcgccctctggtgtgggacctaatacaatggctgcaggcggtggcgcaccaatggcagacaataacgaaggcgccgacggagtgggtagttcctcgggaaattggcattgcgattccacatggctgggcgacagagtcatcaccaccagcacccgaacctgggccctgcccacctacaacaaccacctctacaagcaaatctccaacgggacatcgggaggagccaccaacgacaacacctacttcggctacagcaccccctgggggtattttgactttaacagattccactgccacttttcaccacgtgactggcagcgactcatcaacaacaactggggattccggcccaagagactcagcttcaagctcttcaacatccaggtcaaggaggtcacgcagaatgaaggcaccaagaccatcgccaataacctcaccagcaccatccaggtgtttacggactcggagtaccagctgccgtacgttctcggctctgcccaccagggctgcctgcctccgttcccggcggacgtgttcatgattccccagtacggctacctaacactcaacaacggtagtcaggccgtgggacgctcctccttctactgcctggaatactttccttcgcagatgctgagaaccggcaacaacttccagtttacttacaccttcgaggacgtgcctttccacagcagctacgcccacagccagagcttggaccggctgatgaatcctctgattgaccagtacctgtactacttgtctcggactcaaacaacaggaggcacggcaaatacgcagactctgggcttcagccaaggtgggcctaatacaatggccaatcaggcaaagaactggctgccaggaccctgttaccgccaacaacgcgtctcaacgacaaccgggcaaaacaacaatagcaactttgcctggactgctgggaccaaataccatctgaatggaagaaattcattggctaatcctggcatcgctatggcaacacacaaagacgacgaggagcgtttttttcccagtaacgggatcctgatttttggcaaacaaaatgctgccagagacaatgcggattacagcgatgtcatgctcaccagcgaggaagaaatcaaaaccactaaccctgtggctacagaggaatacggtatcgtggcagataacttgcagcagcaaaacacggctcctcaaattggaactgtcaacagccagggggccttacccggtatggtctggcagaaccgggacgtgtacctgcagggtcccatctgggccaagattcctcacacggacggcaacttccacccgtctccgctgatgggcggctttggcctgaaacatcctccgcctcagatcctgatcaagaacacgcctgtacctgcggatcctccgaccaccttcaaccagtcaaagctgaactctttcatcacgcaatacagcaccggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccgccccattggcacccgttacctcacccgtaatctgtaattgcctgttaatcaataaaccggttgattcgtttcagttgaactttggtctctgcg